(19) United States
(12) Patent Application Publication
(10) Pub. No.: US 2021/0163484 A1
(43) Pub. Date: Jun. 3, 2021

(54) METHOD FOR SYNTHESIZING DIVERSELY SUBSTITUTED PURINES

(71) Applicant: UNIVERSITE GRENOBLE ALPES, Saint Martin d'Hères (FR)

(72) Inventors: Jean-Luc DECOUT, Vaulnaveys Le Haut (FR); Renaud ZELLI, Grenoble (FR); Wael ZEINYEH, Grenoble (FR); Benjamin BOUCHERLE, Saint Martin d'Hères (FR); Romain HAUDECOEUR, Grenoble (FR)

(21) Appl. No.: 16/610,224

(22) PCT Filed: May 3, 2017
(86) PCT No.: PCT/IB2017/000688

§ 371 Date: Nov. 1, 2019

(51) Int. Cl. C07D 473/40; C07H 19/16; C07D 239/48; C07D 239/42

(52) U.S. Cl. CPC C07D 473/40; C07D 239/42; C07D 239/48; C07H 19/16

(57) ABSTRACT

The present invention relates to a method for synthesizing diversely substituted purines starting from a pyrimidine. Formula (I). The method comprises the formation of an amidine group on the pyrimidine by implementing a Vilsmeier type reagent, the functionalization of the pyrimidine with an amine and the cyclization to form the purine nucleus. Optional steps can also be performed in order to further functionalize the molecule. The invention also relates to new purines and new intermediate product.

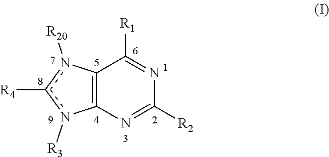

### TECHNICAL FIELD

The present invention relates to a method for synthesizing purines, in particular diversely substituted purines.

### BACKGROUND ART

Purines are heterocyclic compounds comprising a pyrimidine ring fused with an imidazole ring. Purine is a very interesting scaffold that can be found in natural products and in numerous different drugs. For example, the PCT application WO2016/087665 discloses purine derivatives that could be used to treat cystic fibrosis.

Different methods are known and used to synthesize purines. However, these methods can sometimes have a limited scope or lead to poor yields. Thus, there are few methods that can be used in order to introduce a substituent in the position 8 of the purine.

Furthermore, the synthesis of 9-alkylpurines from 9-H-purines is generally done by nucleophilic substitution using an alkyl halide, but this reaction gives a mixture of 7-alkylpurines and 9-alkylpurines due to a selectivity issue.

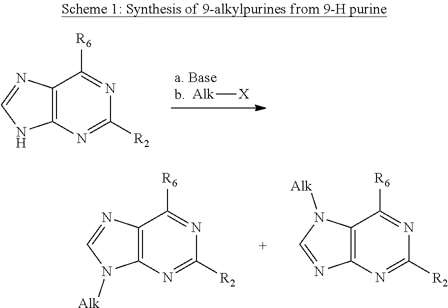

9-aryl purines can be synthesized from 9-H purines using arylboronic acids or aryl halides (first pathway, scheme 2, cf. Niu, H.-Y.; Xia, C.; Qu, G.-R.; Zhang, Q.; Jiang, Y.; Mao, R.-Z.; Li, D.-Y.; Guo, H.-M. Org. Biomol. Chem. 2011, 9, 5039-5042; Foller Larsen, A.; Ulven, T. Chem. Commun. 2014, 50, 4997-4999; Lam, P. Y.; Clark, C. G.; Saubern, S.; Adams, J.; Winters, M. P.; Chan, D. M.; Combs, A. Tetrahedron Lett. 1998, 39, 2941-2944). Nevertheless, with arylboronic acids, the yields are generally low and the scope is limited to available arylboronic acids. With aryl halides, the substituents which are tolerated in the 2, 6 and 8 positions are limited and the introduction of an aryl halide with an electron withdrawing group, like NO₂, results to low yields.

9-arylpurines can also be synthesized from pyrimidine using orthoesters or via a formamidine intermediate (second pathway, scheme 2). These methods also result to low yields when the aniline used is substituted with an electron-withdrawing group, such as NO₂, and/or when halogen atoms are present at position 2 and/or 6 of the purine core.

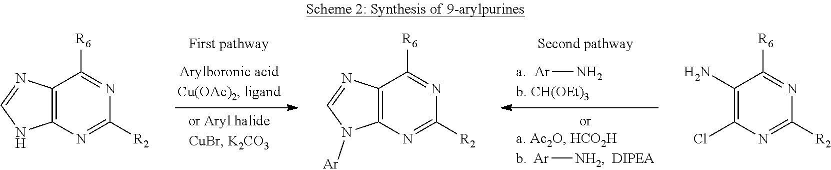

Therefore, there is the need for a versatile method that can be used to synthesize diversely substituted purines with high yields, especially 6-halogenopurines and 2,6-dihalogenopurines that could be further substituted.

### Technical Problem and Objectives to Achieve

In this context, the present invention aims to meet at least one of the objectives stated below.

One of the essential objectives of the present invention is to furnish a method to synthesize purines.

Another essential objective of the present invention is to furnish a versatile method to synthesize diversely substituted purines, especially 6-halogenopurines and 2,6-dihalogenopurines.

Another essential objective of the present invention is to furnish a versatile method to synthesize selectively N7 or N9 substituted purines.

Another essential objective of the present invention is to furnish a versatile method to synthesize 9-arylpurines, especially 9-arylpurines with an electron-withdrawing group on the aryl group.

Another essential objective of the present invention is to furnish a versatile method to synthesize purines functionalized in position 8.

Another essential objective of the present invention is to furnish a method to synthesize diversely substituted purines in high yields, especially in high isolated yields.

Another essential objective of the present invention is to furnish a method to synthesize diversely substituted purines which is easy to implement and uses mild conditions compatible with most of the classical chemical groups.

Another essential objective of the present invention is to furnish new substituted purines.

### SUMMARY

These objectives, among others, are reached by the present invention which first of all relates to a method for synthesizing a purine of formula (I)

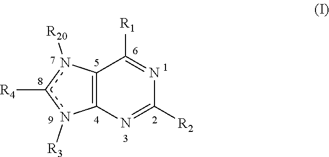

wherein:
the dashed line between the positions 7,8,9 of the imidazole ring symbolizes 2 versions of formula (I): a version v1 wherein there is a double bond between C8 and N7 and hence a radical R₃ on N9 and no radical R₂₀ on N7, as well as a version v2 wherein there is a double bond between C8 and N9 and hence a radical R₂₀ on N7 and no radical R₃ on N9;
R₁ and R₂ are independently selected from the group consisting of hydrogen, halogen, —NR₁₀R₁₁, —N═CR₁₀R₁₁, —OR₁₂, —OSO₂R₁₃, —SOₙR₁₄, —COOR₁₅, —OCOR₁₅, —CONR₁₆R₁₇, alkyl, cycloalkyl, heterocycle, aryl, heteroaryl, alkylaryl, arylalkyl, alkenyl and alkynyl, n being a number between 0 and 2;
   R₁₀, R₁₁, R₁₂, R₁₃, R₁₄, R₁₅, R₁₆ and R₁₇ being selected independently from the group consisting of hydrogen, alkyl, cycloalkyl, dialkylamino, heterocycle, aryl, heteroaryl, alkylaryl, arylalkyl, alkenyl and alkynyl;
R₃, if present, is selected from the group consisting of R₁₈ and R₁₉,
   R₁₈ being selected from the group consisting of glycosyl,
   R₁₉ being selected from the group consisting of hydrogen, alkyl, cycloalkyl, heterocycle, aryl, heteroaryl, alkylaryl, arylalkyl, alkenyl, alkynyl, trialkylsilyl, triarylsilyl, and trialkylarylsilyl;
R₄ is selected from the group consisting of R₆, R₇ and NR₈R₉;
   R₆ and R₇ being independently selected from the group consisting of hydrogen, alkyl, cycloalkyl, heterocycle, aryl, heteroaryl, alkylaryl, arylalkyl, alkenyl, alkynyl, halogen, azido, —OR₃₁ and —NR₁₀R₁₁;
   R₃₁ being selected from the group consisting of hydrogen, —COR₃₂, —SOₙR₃₃, hydrogen, alkyl, cycloalkyl, heterocycle, aryl, heteroaryl, alkylaryl, arylalkyl, alkenyl, alkynyl, trialkylsilyl, triarylsilyl, and trialkylarylsilyl, n being a number between 0 and 2;
   R₈ and R₉ being independently selected from the group consisting of hydrogen, alkyl, cycloalkyl, heterocycle, aryl, heteroaryl, alkylaryl, arylalkyl, alkenyl and alkynyl, or R₈ and R₉ are linked to form a ring;
   R₃₂ and R₃₃ being independently selected from the group consisting of hydrogen, alkyl, cycloalkyl, heterocycle, aryl, heteroaryl, alkylaryl, arylalkyl, alkenyl and alkynyl;
   R₁₀ and R₁₁ are as defined above;
R₂₀, if present, is selected from the group consisting of R₁₈ and R₁₉,
   R₁₈ and R₁₉ are as defined above;
said method consisting in starting from a pyrimidine of formula (II)

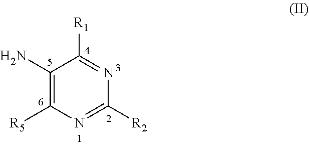

wherein
R₁ and R₂ are as defined above;
R₅ is selected from the group consisting of halogen, NHR₁₀, azido and —OR₃₁, R₁₀ and R₃₁ being as defined above;
said method comprising the following steps a), optionally step b), optionally step c), optionally step d), in any order, and then step e)
   a) formation of an amidine group at the C5 or C6 position of the pyrimidine by implementing a Vilsmeier type reagent of formula (III) and/or a reagent of formula (IV)

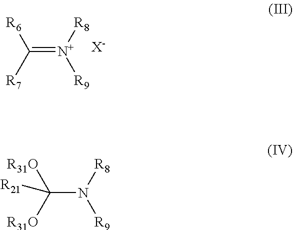

wherein
   X⁻ is a counterion;
   R₂₁ is selected from the group consisting of R₆ and R₇;
   R₆, R₇, R₈, R₉ and R₃₁ are as defined above;
b) optionally, substitution of R₅ by an amine of formula NH₃ or NHR₁₉R₃₄, wherein:
   R₁₉ is as defined above,
   R₃₄ is selected from the group consisting of hydrogen, —COR₃₅, —SOₙR₃₆, alkyl, cycloalkyl, heterocycle, aryl, heteroaryl, alkylaryl, arylalkyl, alkenyl and alkynyl; and n being a number between 0 and 2;
      R₃₅ and R₃₆ being selected from the group consisting of hydrogen, alkyl, cycloalkyl, heterocycle, aryl, heteroaryl, alkylaryl, arylalkyl, alkenyl and alkynyl;
c) optionally, substitution of Y, in a reagent of formula R₁₈Y, by the amino group —NH₂ or —NR₁₉R₃₄ at the C6 position of the pyrimidine, wherein:
   R₁₈ is as defined above,
   Y is selected from the group consisting of halogen, azido and —OR₃₇, R₃₇ being selected from the group consisting of hydrogen, —COR₃₈, —SOₙR₃₉, alkyl, cycloalkyl, heterocycle, aryl, heteroaryl, alkylaryl, arylalkyl, alkenyl and alkynyl, and n being a number between 0 and 2
      R₃₈ and R₃₉ being selected from the group consisting of hydrogen, alkyl, cycloalkyl, heterocycle, aryl, heteroaryl, alkylaryl, arylalkyl, alkenyl and alkynyl;
d) optionally, functionalization of the —NH₂ at the C5 position of the pyrimidine of formula (II), leading to a —NHR₂₀ group, R₂₀ being as defined above;
e) cyclization to form the purine nucleus of formula (I);
with the proviso that when R₈ is NH₂, R₁ is not OH and R₂ is not NH₂;
and wherein, in the above definitions of formulae (I), (II), (III) & (IV), any alkyl, cycloalkyl, heterocycle, aryl, heteroaryl, alkylaryl, arylalkyl, alkenyl or alkynyl group is optionally substituted with one or more substituents selected from the group consisting of alkyl, halogen, haloalkyl, alkoxy, hydroxy, acyl, alkyloxycarbonyl, amino, imino, nitro, —SO₃H, —COOH, —CONH₂, cyano, thiol and oxo.

This versatile method gives access to diversely substituted purines, especially purines with substituents in positions 2, 6, 7, 8 and/or 9, in moderate to excellent yields. In particular, 6-halogenopurines and 2,6-dihalogenopurines can be synthesized in good yields. These types of purines are interesting as they can easily be further functionalized.

The use of a Vilsmeier type reagent of formula (III) and/or a reagent of formula (IV) allows for an easy introduction of a substituent in position 8 of the purine. Furthermore, a variety of different substituents can be introduced in position 9. For example, 9-arylpurines with an electron-withdrawing group on the aryl can be synthesized in good yields.

This method allows on the one hand, the selective production of N-9-alkyl- and aryl-purines without formation of N-7-alkyl and -arylpurines as by-products; and on the other hand the selective synthesis of N-7-alkyl- and arylpurines without formation of N-9-alkyl- and -arylpurines as by-products.

Moreover, this method can be carried out in mild conditions and possibly in one pot, there is no need for purification of the different intermediates. The mild conditions are compatible with a variety of functional groups on the reagents.

This method can also be used to synthesize new purines of potential biological or medicinal interest.

### Definitions

The term “substituted” refers to an organic group as defined herein or a molecule in which one or more atoms are replaced with one or more substituents different from hydrogen atoms.

The term “halogen” refers to fluoro (—F), chloro (—Cl), bromo (—Br), or iodo (—I) groups. Preferred halogen groups are fluoro and chloro.

The term “alkyl” by itself or as part of another substituent refers to a hydrocarbyl radical of formula CₙCH₂ₙ₊₁ wherein n is a number greater than or equal to 1. Generally, alkyl groups of this invention comprise from 1 to 12 carbon atoms, preferably from 1 to 6 carbon atoms, more preferably from 1 to 4 carbon atoms. Alkyl groups may be linear or branched and may be substituted as indicated herein. Suitable alkyl groups include methyl, ethyl, n-propyl, i-propyl, n-butyl, i-butyl, s-butyl and t-butyl, pentyl and its isomers (e.g. n-pentyl, iso-pentyl), and hexyl and its isomers (e.g. n-hexyl, iso-hexyl).

The term “cycloalkyl” refers to a cyclic hydrocarbyl radical. Generally, cycloalkyl group of this invention comprise from 3 to 12 carbon atoms, preferably from 3 to 6 carbon atoms. Suitable cycloalkyl groups include cyclopropyl, cyclobutyl, cyclopentyl and cyclohexyl.

The term “heterocycle” refers to a cyclic compound having as ring members, atoms of at least two different elements, like carbon and oxygen atoms, or carbon and nitrogen atoms, or carbon and sulfur atoms. Generally, heterocycle group of this invention comprise from 3 to 7 ring atoms. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The term “aryl” refers to a polyunsaturated, aromatic hydrocarbyl group having a single ring (i.e. phenyl) or multiple aromatic rings fused together (e.g. naphtyl), typically containing 5 to 12 atoms; preferably 6 to 10, wherein at least one ring is aromatic. The aromatic ring may optionally include one to two additional rings (cycloalkyl, heterocyclyl or heteroaryl) fused thereto.

The term “heteroaryl” refers to aromatic rings or ring systems containing 1 to 2 rings which are fused together or linked covalently, typically containing 5 to 12 atoms or 5 to 6 atoms; at least one of which is aromatic, in which one or more carbon atoms in one or more of these rings is replaced by oxygen, nitrogen and/or sulfur atoms where the nitrogen and sulfur heteroatoms may optionally be oxidized and the nitrogen heteroatoms may optionally be quaternized. Such rings may be fused to an aryl, cycloalkyl, heteroaryl or heterocyclyl ring. Non-limiting examples of such heteroaryl, include: furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, indolyl, isoindolyl, benzofuranyl, isobenzofuranyl, benzothiophenyl, isobenzothiophenyl, indazolyl, benzimidazolyl, benzoxazolyl, purinyl, benzodioxolyl, quinolinyl, isoquinolinyl, cinnolinyl, quinazolinyl and quinoxalinyl.

The term “arylalkyl” refers to a moiety aryl-alkyl-, wherein aryl and alkyl are as defined above. Arylalkyl groups include, for example, benzyl groups.

The term “alkylaryl” refers to a moiety alkyl-aryl-, wherein alkyl and aryl are as defined above.

The term “haloalkyl” refers to an alkyl as defined above wherein one or more hydrogen atoms on the alkyl is replaced by a halogen group defined above.

The term “alkoxy” refers to an alkyl-O— group in which the alkyl group is as previously described. Non-limiting examples of suitable alkoxy groups include methoxy, ethoxy, propoxy, isopropoxy and butoxy.

The “alkyloxycarbonyl” refers to an alkyl group as defined above singularly bonded to an oxy carbonyl group (O—CO—). Alkyloxycarbonyl include, for example —O—CO—CH₃, —O—CO—C₂H₅, —O—CO—C₄H₉.

The term“alkenyl” as used herein refers to a monovalent group derived from a C₁-C₁₂ inclusive straight or branched hydrocarbon moiety having at least one carbon-carbon double bond. Alkenyl groups include, for example, ethenyl (i.e., vinyl), propenyl, butenyl, 1-methyl-2-buten-1-yl, pentenyl, hexenyl, octenyl, and butadienyl.

The term “alkynyl” as used herein refers to a monovalent group derived from a straight or branched C₁-C₁₂ hydrocarbon of a designed number of carbon atoms containing at least one carbon-carbon triple bond. Examples of “alkynyl” include ethynyl, 2-propynyl (propargyl), 1-propynyl, pentynyl, hexynyl, heptynyl, and allenyl groups, and the like.

The expression “between x and y” is understood to mean a range or ranges of values, the limits whereof are closed: [x,y].

The terms “electron-withdrawing group” refers to an individual atom or a functional group that withdraws electron density from a conjugated system.

The term “anhydrous” refers to a solvent having less than 100 ppm water, preferably less than 50 ppm.

The term “glycosyl” refers to a structure obtained by removing the hydroxy group from the hemiacetal function of a monosaccharide and, by extension, of an oligosaccharide or a polysaccharide (modified saccharides such as pseudo-oligosaccharides are also included in this definition).

### DETAILED DESCRIPTION

### Synthesis of Purines of Formula (I)

The invention relates to a method for synthesizing purines of formula (I) starting from a pyrimidine of formula (II).

According to one embodiment of the invention, R₁ is selected from the group consisting of hydrogen, halogen, —NR₁₀R₁₁, —OR₁₂, alkyl, heterocycle, aryl, heteroaryl, alkylaryl and arylalkyl;
R₁₀, R₁₁ and R₁₂, being selected independently from the group consisting of hydrogen, and alkyl.

R₁ can be a halogen, preferably —Cl.

According to one embodiment of the invention, R₂ is selected from the group consisting of hydrogen, halogen, —NR₁₀R₁₁, —N═CR₁₀R₁₁, —OR₁₂, alkyl, heterocycle, aryl, heteroaryl, alkylaryl and arylalkyl;
R₁₀, R₁₁ and R₁₂, being selected independently from the group consisting of hydrogen, dialkylamino, and alkyl.

R₂ can be selected from the group consisting of hydrogen, halogen, preferably —Cl, —NH2, and alkyl, preferably methyl.

According to one embodiment of the invention, R₃ is selected from the group consisting of R₁₈ and R₁₉,
R₁₈ being selected from the group consisting of glycosyl,
R₁₉ being selected from the group consisting of hydrogen, alkyl, cycloalkyl, arylalkyl, alkylaryl and aryl.

According to another embodiment, R₃ is R₁₈
R₁₈ being selected from the group consisting of ribosyl and desoxyribosyl, preferably 2′-deoxyribosyl.

According to one embodiment, R₃ is R₁₉,
R₁₉ being selected from the group consisting of alkyl, arylalkyl, heteroaryl, and aryl. Preferably the aryl is substituted with at least one electron withdrawing group like halogen, nitro, cyano, —SO₃H, —COOH or —CONH₂.

According to one embodiment of the invention, R₄ is selected from the group consisting of R₆, R₇ and NR₈R₉;
R₆ and R₇ being independently selected from the group consisting of hydrogen, halogen, alkyl, preferably methyl, and aryl, preferably phenyl;
R₈ and R₉, being independently selected from the group consisting of hydrogen and alkyl, preferably methyl;

According to one embodiment of the invention, step a) is performed by implementing a Vilsmeier type reagent of formula (III) and/or a reagent of formula (IV)

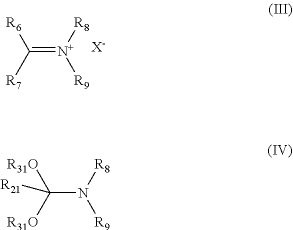

wherein
   X⁻ is a counterion;
   R₂₁ is selected from the group consisting of R₆ and R₇;
   R₆ and R₇ are independently selected from the group consisting of hydrogen, halogen, alkyl, preferably methyl, and aryl, preferably phenyl;
   R₈ and R₉ are independently selected from the group consisting of hydrogen and alkyl, preferably methyl;
   R₃₁ is selected from the group consisting of alkyl, preferably methyl;

According to one embodiment of the invention, R₆ or R₇ is a halogen, preferably —Cl.

According to one embodiment, R₈ and R₉ are alkyl groups, preferably methyl groups.

For example, the counterion X⁻ is a sulfonate, BF₄⁻, PF₆⁻, or a halide, preferably a halide, more preferably Cl⁻.

According to one embodiment of the invention, R₂₀ is selected from the group consisting of R₁₈ and R₁₉,
R₁₈ being selected from the group consisting of glycosyl,
R₁₉ being selected from the group consisting of hydrogen, alkyl, cycloalkyl, alkylaryl, arylalkyl and aryl.

According to another embodiment, R₂₀ is R₁₈
R₁₈ being selected from the group consisting of ribosyl and desoxyribosyl, preferably 2′-deoxyribosyl.

According to one embodiment, R₂₀ is R₁₉,
R₁₉ being selected from the group consisting of alkyl, arylalkyl, heteroaryl, and aryl. Preferably the aryl is substituted with at least one electron withdrawing group like halogen, nitro, cyano, —SO₃H, —COOH or —CONH₂.

According to one embodiment of the invention, R₅ is selected from the group consisting of halogen, preferably —Cl.

According to one embodiment of the invention, Y is selected from the group consisting of halogen, azido and —OR₃₇, R₃₇ being —COR₃₈, R₃₈ being a methyl group.

According to a preferred embodiment, the present invention relates to a method for synthesizing a purine of formula (I)

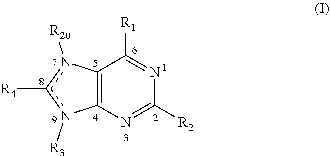

wherein:
the dashed line between the positions 7,8,9 of the imidazole ring symbolizes 2 versions of formula (I): a version v1 wherein there is a double bond between C8 and N7 and hence a radical R₃ on N9 and no radical R₂₀ on N7, as well as a version v2 wherein there is a double bond between C8 and N9 and hence a radical R₂₀ on N7 and no radical R₃ on N9;
R₁ and R₂ are independently selected from the group consisting of hydrogen, halogen, —NR₁₀R₁₁ and alkyl;
R₁₀ and R₁₁, being selected independently from the group consisting of hydrogen and alkyl;
R₃, if present, is selected from the group consisting of R₁₈ and R₁₉,
   R₁₈ being selected from the group consisting of glycosyl,
   R₁₉ being selected from the group consisting of alkyl, arylalkyl and substituted aryl, preferably the substituted aryl is substituted with at least one electron withdrawing group like halogen, nitro, cyano, —SO₃H, —COOH or —CONH₂;
R₄ is selected from the group consisting of R₆, R₇ and NR₈R₉;
   R₆ and R₇ being independently selected from the group consisting of hydrogen, halogen, and aryl, preferably phenyl;
   R₈ and R₉, being independently selected from the group consisting of hydrogen and alkyl, preferably methyl;
R₂₀ if present, is selected from the group consisting of R₁₈ and R₁₉,
   R₁₈ being selected from the group consisting of glycosyl,
   R₁₉ being selected from the group consisting of alkyl, arylalkyl and substituted aryl, preferably the substituted aryl is substituted with at least one electron withdrawing group like halogen, nitro, cyano, —SO₃H, —COOH or —CONH₂.

The above described embodiment can be combined with one another.

### Steps a), b), c), d) and e)

The invention relates to a method for synthesizing purines of formula (I) as mentioned above. The method comprises steps a), optionally step b), optionally step c), optionally step d), in any order, and then step e).

Step e) is always the last step. Steps a), b), c) and d) can be performed in any order. According to a preferred embodiment, the method does not comprise both steps c) and d): if step c) is performed, step d) is not and if step d) is performed, step c) is not. According to one embodiment, step a) is performed before step b). According to another embodiment, step b) is performed before step a).

According to a first embodiment, the method comprises steps a), b), in any order, and step e). This first embodiment is particularly suited to synthesize diversely substituted 9-arylpurine, especially with an electron-withdrawing group on the aryl.

According to this first embodiment, first step a) can be performed, then step b) and finally step e). In this case, step a) is performed at the C5 position.

According to this first embodiment, another possibility is that first step b) is performed, then step a) and finally step e). In this case, step a) is generally performed on the C5 position.

An example of the method according to this first embodiment is presented on scheme 3.

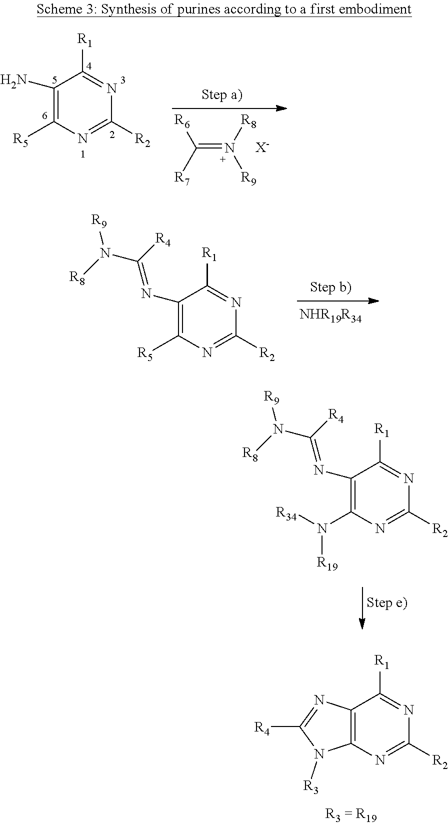

According to a second embodiment, the method comprises steps a), b), c), in any order, and step e). In this case, step a) occurs at the C5 position of the pyrimidine and step b) is performed before step c). This second embodiment is particularly suited to synthesize diversely substituted purine nucleosides when R₁ is a glycosyl.

According to this second embodiment, first step a) can be performed, then step b), then step c) and finally step e).

According to this second embodiment, another possibility is that first step b) is performed, then step a), then step c) and finally step e).

According to this second embodiment, another possibility is that first step b) is performed, then step c), then step a) and finally step e).

An example of the method according to this second embodiment is presented on scheme 4.

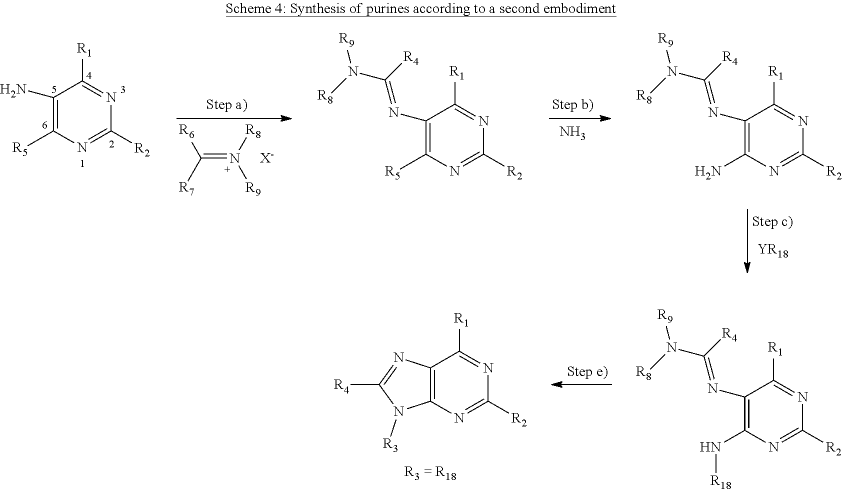

According to a third embodiment, the method comprises steps a), b), d), in any order, and step e). In this case, step a) occurs at the C6 position of the pyrimidine and step b) has to be performed before step a). This third embodiment is particularly suited to synthesize diversely substituted 7-alkyl- or 7-aryl-purines.

According to this third embodiment, first step b) can be performed, then step a), then step d) and finally step e).

According to this third embodiment, another possibility is that first step b) is performed, then step d), then step a) and finally step e).

According to a third embodiment, first step d) is performed, then step b), then step a) and finally step e).

An example of the method according to this third embodiment is presented on scheme 5.

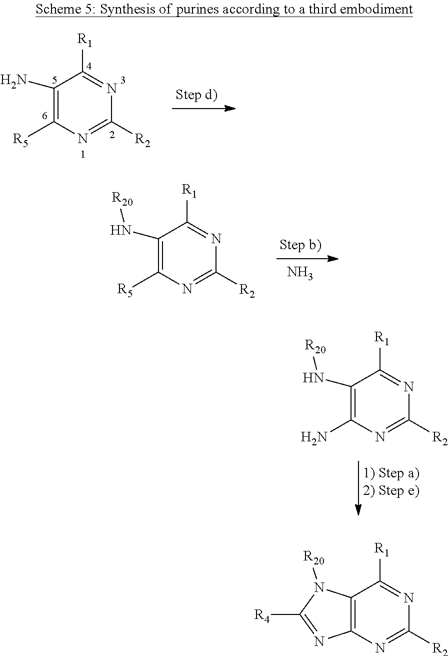

According to a fourth embodiment, the method comprises step a) and then step e). In this case, step a) occurs preferably at the C5 position of the pyrimidine and R₅ is NHR₁₀.

An example of the method according to this fourth embodiment is presented on scheme 6.

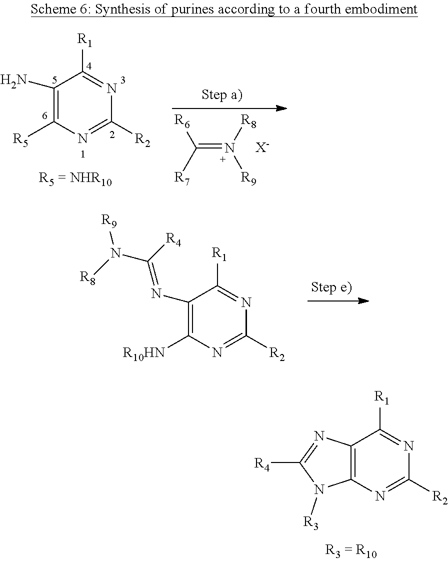

According to a fifth embodiment, the method comprises steps a), c), in any order, and step e). In this case, step a) occurs at the C5 position of the pyrimidine and R₅ is NH₂.

This fifth embodiment is particularly suited to synthesize diversely substituted purine nucleosides when R₁₈ is a glycosyl.

According to this fifth embodiment, first step a) can be performed, then step c) and finally step e).

According to this fifth embodiment, another possibility is that first step c) is performed, then step a) and finally step e).

An example of the method according to this second embodiment is presented on scheme 7.

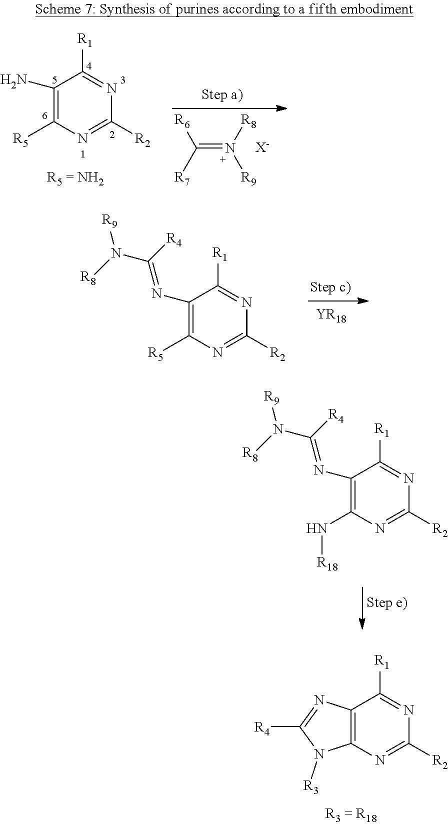

According to a sixth embodiment, the method comprises steps a), d), in any order, and step e). In this case, step a) occurs at the C6 position of the pyrimidine and R₅ is NH₂.

This sixth embodiment is particularly suited to synthesize diversely substituted 7-alkyl- or 7-aryl-purines.

According to this sixth embodiment, first step a) can be performed, then step d) and finally step e).

According to this sixth embodiment, another possibility is that first step d) is performed, then step a) and finally step e).

An example of the method according to this third embodiment is presented on scheme 8.

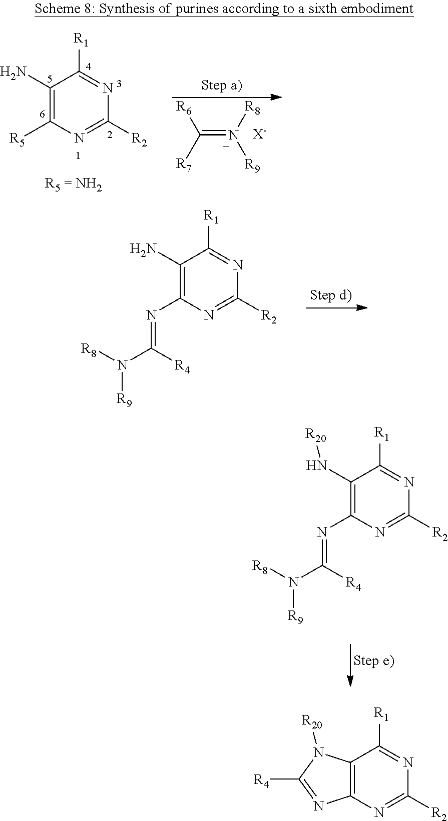

### Method for Synthesizing Purines of Formula (I)

According to one embodiment of the invention, the method as described above also comprises a step f), performed after step e), of isolation of the purine of formula (I).

According to one embodiment, the method is performed under inert atmosphere, for example, under argon or nitrogen.

According to one embodiment, the method is performed using Brønsted or Lewis acid catalyst and/or by heating the reaction mixture. For example, step b) and/or step c) and/or step e) is performed using Brønsted or Lewis acid catalyst and/or by heating the reaction mixture. Examples of Brønsted acid catalyst include, but are not limited to, p-toluenesulfonic acid (APTS) and benzenesulfonic acid. Examples of Lewis acid catalyst include, but are not limited to zinc chloride and tin (IV) chloride.

The reaction mixture can be heated at a temperature comprised between room temperature (r.t.) and the reflux temperature of the solvent used for performing the reaction. The reaction mixture can be heated using a microwave.

According to a preferred embodiment, the method is performed in an organic solvent. For example, the reaction is performed in dioxane, 1,2-dichloroethane, alcools, DMF or 1,2-dimethoxyethane.

The method can be performed in one pot, which means with no purification of intermediates products. For example, according to the first embodiment, steps b), a) and then e) can be performed in one pot. It is also possible to perform steps c) and e) or steps b) and e) in one pot. However, it is also possible to isolate the intermediates like, for example, the amidine after step a), the pyrimidine with a substituted amine on C6 after step b) or the pyrimidine with a substituted amine on C5 after step d).

The Vilsmeier reagent of formula (III) used in step a) can be synthesized in situ. For example it can be synthesized from the corresponding amide and a reagent selected from POCl₃, oxalyl chloride and SOCl₂.

According to one embodiment, the reaction is performed in anhydrous conditions, which means in an anhydrous solvent. For example, steps a) and b) can be performed in anhydrous conditions.

Step d) can be performed according to any known conventional method used to functionalize amine. For example, reductive amination or nucleophilic substitution can be used to functionalize a —NH₂ group at the C5-position of the pyrimidine.

### Purines of Formula (I) and Intermediate Products

The invention also relates to products obtainable by the process as described above.

The invention also relates to new intermediates products of the process as described above, especially intermediate products selected form the group consisting of:

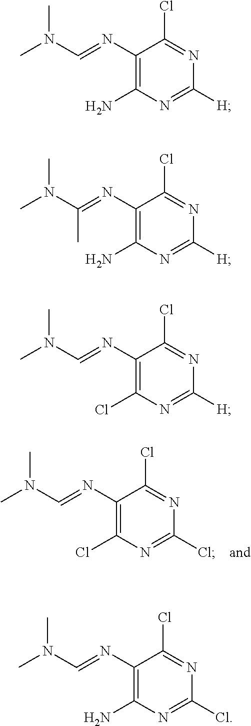

The invention also relates to new substituted purines of formula (I), especially purines selected from the group consisting of:

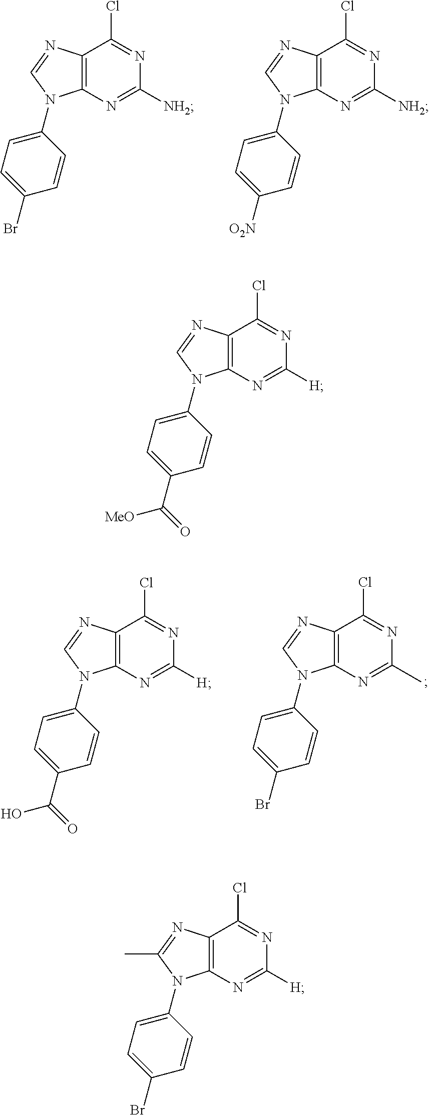

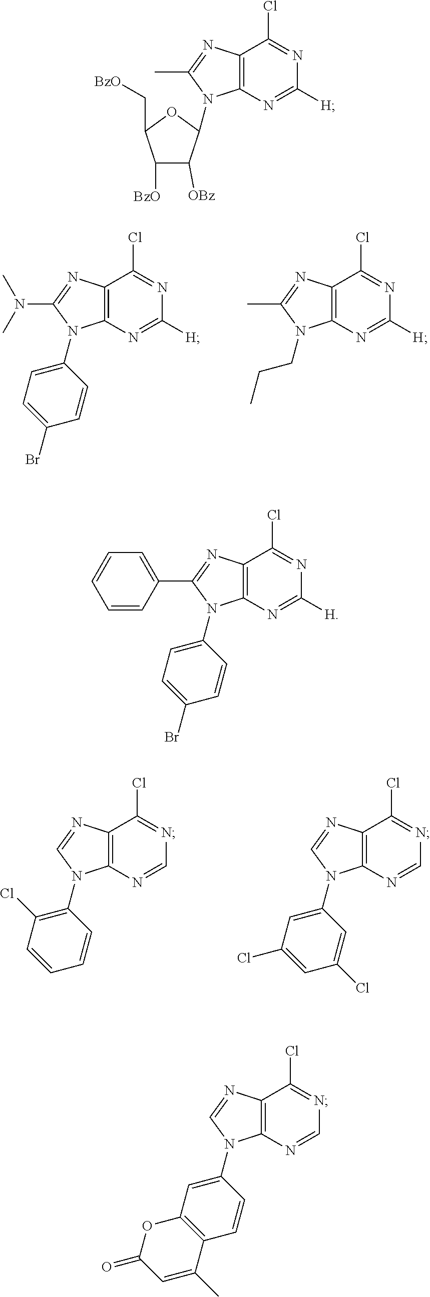

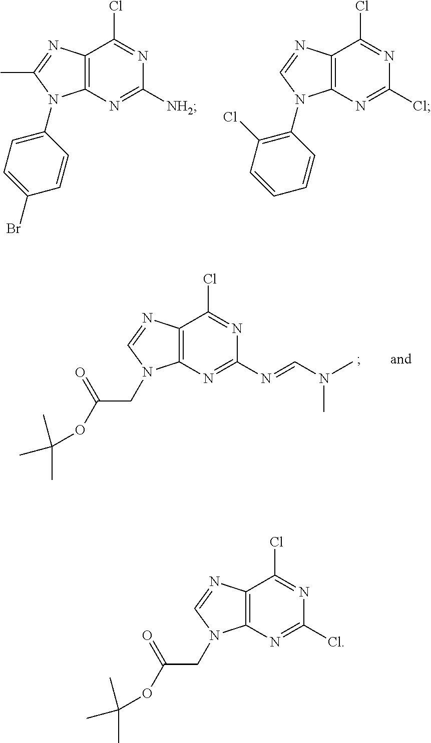

These purines are obtainable by the process as described above.

The present invention also relates to pharmaceutically acceptable salt of these molecules and their preparation.

### Examples

Materials

All starting materials were commercially available research grade chemicals and used without further purification. They were purchased from Sigma-Aldrich, Fisher, Tokyo Chemical Industry, or Alfa Aesar. Reactions were monitored by analytical TLC on silica gel (Alugram Sil G/UV254) from Macherey-Nagel with fluorescent indicator UV254. HRMS analyses were obtained from the Mass Spectrometry Service, ICOA, at the University of Orléans, France, using a HRMS Q-Tof MaXis spectrometer. ¹H and ¹³C NMR spectra were recorded on a Bruker Avance 400 at 400 MHz and 100 MHz respectively using the residual solvent signal as internal standard. Chemical shifts are reported in ppm (parts per million) relative to the residual signal of the solvent, and the signals are described as singlet (s), broad singlet (bs), doublet (d), triplet (t), doublet of doublet (dd), quartet (q), sextuplet (sext), septuplet (sept), multiplet (m); coupling constants are reported in Hertz (Hz). Column chromatographies were performed on silica gel (MN Kieselgel 60, 0.063e0.2 mm/70e230 mesh, Machereye-Nagel) or on C18 reversed phase (Macherey-Nagel Polygoprep 60e50 C18).

All equivalents are expressed in mol.

### General Methods of Synthesis

### General Synthesis Protocol I

To a solution of pyrimidine (100 mg, 1 equivalent) in anhydrous dioxane (2 mL/mmol) under argon atmosphere, were successively added the amine (1 equivalent) and APTS.H₂O (0.5 equivalent). The solution was stirred at reflux for 18 h and then allowed to reach room temperature. A 0.2 M solution of corresponding dialkyliminium chloride (1.5 to 3 equivalents) in anhydrous DMF, or a 0.2 M solution of POCl₃ or oxalyl chloride (1.5 to 3 equivalents) in the corresponding N,N-dialkylamide was then added dropwise and the resulting mixture was stirred for another 30 min. The solution was diluted in DCM or AcOEt (5 mL/mmol) and washed with a saturated aqueous solution of NaHCO₃ (5 mL/mmol). The aqueous layer was extracted 3 times with DCM or AcOEt and the resulting organic layer was then dried over magnesium sulfate. After concentration under reduced pressure, the crude product was purified by chromatography on silica gel to afford the pure compound.

### General Synthesis Protocol II

To a solution of pyrimidine (100 mg, 1 equivalent) in anhydrous dioxane (2 mL/mmol) under argon atmosphere, were successively added the amine (1 equivalent) and APTS.H₂O (0.5 equivalent). The solution was stirred at reflux for 6 h and then allowed to reach room temperature. A 0.2 M solution of corresponding dimethyliminium chloride (2 to 3 equivalents) in anhydrous DMF, or a 0.2 M solution of POCl₃ or oxalyl chloride (1.5 to 3 equivalents) in corresponding N,N-dimethylamide was then added dropwise and the resulting mixture was stirred for another hour. Water (5 mL/mmol) was then slowly added and the resulting mixture was stirred at room temperature for 18 h. The solution was diluted in AcOEt (5 mL/mmol) and washed with a saturated aqueous solution of NaHCO₃ (5 mL/mmol). The aqueous layer was extracted 3 times with AcOEt and the resulting organic layer was then dried over magnesium sulfate.

After concentration under reduced pressure, the crude product was purified by chromatography on silica gel to afford the pure compound.

### General Synthesis Protocol III

To a solution of pyrimidine (100 mg, 1 equivalent) in n-butanol (2 mL/mmol) were successively added the amine (1 equivalent) and DIPEA (2 equivalents). The solution was stirred at reflux for 18 h and then concentrated under reduced pressure. A 0.2 M solution of corresponding dimethyliminium chloride (2 to 3 equivalents) in anhydrous DMF, or a 0.2 M solution of POCl₃ or oxalyl chloride (1.5 to 3 equivalents) in corresponding N,N-dimethylamide was then added dropwise and the resulting mixture was stirred for another 2 h at 40° C. The solution was diluted in DCM or AcOEt (5 mL/mmol) and washed with a saturated aqueous solution of NaHCO₃ (5 mL/mmol). The aqueous layer was extracted 3 times with DCM or AcOEt and the resulting organic layer was then dried over magnesium sulfate. After concentration under reduced pressure, the crude product was purified by chromatography on silica gel to afford the pure compound.

### General Synthesis Protocol IV

To a solution of pyrimidine (100 mg, 1 equivalent) in anhydrous dioxane (2 mL/mmol) under argon atmosphere, were successively added the amine (1 equivalent) and APTS.H₂O (0.5 equivalent). The solution was stirred at reflux for 18 h and then allowed to reach room temperature. DIPEA (2 equivalents) was then added and the solution was concentrated under reduced pressure. A 0.2 M solution of corresponding dimethyliminium chloride (2 to 3 equivalents) in anhydrous 1,2-dichloroethane, or a 0.2 M solution of POCl₃ or oxalyl chloride (2 to 3 equivalents) and corresponding N,N-dimethylamide (2 to 3 equivalents) was then added to the residue and the resulting mixture was stirred for another 1 h at 80° C. The solution was diluted in DCM or AcOEt (5 mL/mmol) and washed with a saturated aqueous solution of NaHCO₃ (5 mL/mmol). The aqueous layer was extracted 3 times with DCM or AcOEt and the resulting organic layer was then dried over magnesium sulfate. After concentration under reduced pressure, the crude product was purified by chromatography on silica gel to afford the pure compound.

### General Synthesis Protocol V

To a solution of the corresponding amidine (100 mg, 1 equivalent) in anhydrous 1,2-dichloroethane (10 mL/mmol) under argon atmosphere, were successively added 1-O-acetyl-2,3,5-tri-O-benzoyl-β-D-ribose (1 equivalent) and TMSOTf (1 equivalent). The resulting mixture was heated to reflux for 16 h. The solution was diluted in DCM (20 mL/mmol) and washed with a saturated aqueous solution of NaHCO₃ (20 mL/mmol). The aqueous layer was extracted 3 times with DCM and the resulting organic layer was then dried over magnesium sulfate. After concentration under reduced pressure, the crude product was purified by chromatography on silica gel to afford the pure compound.

### Synthesis of Intermediates

### Intermediate Int-1

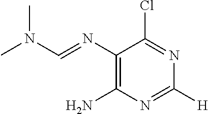

A solution of 6-chloro-4,5-diaminopyrimidine (50 mg, 0.34 mmol) in anhydrous DMF (1 mL) under argon atmosphere was stirred at 0° C. for 10 min before addition of POCl₃ (33 μL). After 2 h of stirring at room temperature, a solution of saturated NaHCO₃ was added and the resulting mixture was extracted 3 times with AcOEt, dried over MgSO₄ and concentrated in vacuo. The residue was then purified by chromatography on silica gel (eluent CH₂Cl₂/MeOH) to afford pure compound Intermediate Int-1 (60%).

¹H NMR (400 MHz, MeOD) δ 7.86 (s, 1H, CHₐᵣ), 7.65 (s, 1H, CHₐᵣ), 3.07 (2s, 6H, 2 CH₃). ¹³C NMR (100 MHz, MeOD) δ 161.9 (C), 159.3 (CH), 151.9 (CH), 147.1 (C), 128.8 (C), 40.6 (CH₃), 34.5 (CH₃). HRMS (ESI) calc. for C₇H₁₁ClN₅: [M+H]⁺ 200.0697, found 200.0697.

### Intermediate Int-2

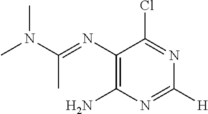

A solution of 6-chloro-4,5-diaminopyrimidine (50 mg, 0.34 mmol) in anhydrous N,N-dimethylacetamide (1 mL) under argon atmosphere is stirred at 0° C. for 10 min before addition of POCl₃ (99 μL). After another 2 hours of stirring at r. t., a solution of sat. NaHCO₃ is added and the resulting mixture is extracted 3 times with AcOEt, dried over MgSO₄ and concentrated in vacuo. The residue is then purified by chromatography on silica gel (eluent CH₂Cl₂/MeOH) to afford pure compound Intermediate Int-2 (80%).

¹H NMR (400 MHz, MeOD) δ 7.89 (s, 1H, CHₐᵣ), 3.11 (s, 6H, 2 CH₃), 1.83 (s, 3H, CH₃). ¹³C NMR (100 MHz, MeOD) δ 163.6 (C), 161.3 (C), 151.8 (CH), 148.1 (C), 128.1 (C), 38.8 (2 CH₃), 16.0 (CH₃). HRMS (ESI) calc. for C₈H₁₃ClN₅: [M+H]⁺ 214.0854, found 214.0856.

### Intermediate Int-3

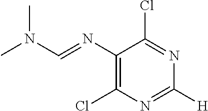

To a 0° C. solution of 5-amino-4,6-dichloropyrimidine (250 mg, 1.52 mmol) in dry DMF (8 mL) under argon atmosphere was added SOCl₂ (328 μL, 4.58 mmol). After stirring 1 h at room temperature, EtOH (4 mL) was added and the resulting solution was concentrated in vacuo. The residue was solubilized in water and a saturated solution of NaHCO₃ was added slowly. The aqueous layer was extracted 3 times with AcOEt and the resulting organic layer was dried over MgSO₄ and concentrated in vacuo to afford pure Intermediate Int-3 (100%) as a pale marroon solid.

¹H NMR (400 MHz, CDCl₃) δ 8.34 (s, 1H, CH_{Ar}), 7.53 (s, 1H, CHN), 3.11 (s, 3H, CH₃), 3.09 (s, 3H, CH₃). ¹³C NMR (100 MHz, CDCl₃) δ 155.6 (C), 153.8 (C), 149.7 (CH), 141.8 (C), 40.5 (CH₃), 34.4 (CH₃). HRMS (ESI) calc. for C₇H₉C₂N₄: [M+H]⁺ 219.0199 found 219.0200.

### Intermediate Int-4

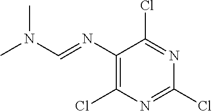

To a suspension of N-(2-amino-4,6-dichloropyrimidin-5-yl)-N′,N′-dimethylimidoformamide (1.5 g, 6.4 mmol) and benzyltriethylammonium chloride (6.6 g, 28.9 mmol) in 1,2-dichloroethane (50 mL) was added tert-butyl nitrite (7.7 mL, 64 mmol), and the mixture was heated to 70° c. under argon atmosphere for 1 h 30. After cooling, the solvent was evaporated under reduced pressure, and the residue was taken up in CH₂Cl₂ and water. The organic layer was washed twice with water, dried over MgSO₄, then concentrated. The residue was purified by chromatography on silica gel (eluent:cyclohexane/ethylacetate, 10/0 to 9/1) to afford Intermediate Int-4 as a yellowish solid (0.7 g, 44%).

¹H NMR (400 MHz, d₆-DMSO) δ 7.87 (s, 1H), 3.06 (s, 3H), 2.99 (s, 3H); ¹³C NMR (125 MHz, d₆-DMSO) δ 156.7 (CH), 153.8 (C), 147.0 (C), 141.2 (C), 39.8 (CH₃), 33.7 (CH₃). HRMS (ESI) calc. for C₇H₈Cl₃N4: [M+H]⁺ 252.9809 found 252.9809.

### Intermediate Int-5

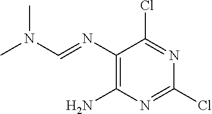

A suspension of Intermediate Int-4 (200 mg, 0.79 mmol) in a mixture of isopropanol (15 mL) and 25% aqueous ammonia (25 mL) was stirred overnight at room temperature. Isopropanol was then evaporated, and the aqueous layer was extracted with ethyl acetate. The organic layer was dried over MgSO₄ then concentrated. The residue was purified by chromatography on silica gel (eluent: cyclohexane/ethyl acetate, 7/3 then 5/5) to afford Intermediate Int-5 (150 mg, 81%). ¹H NMR (400 MHz, d₆-DMSO) δ 7.69 (s, 1H, CH), 7.58 (bs, 1H, NH), 6.74 (bs, 1H, NH), 2.99 (s, 3H, CH₃), 2.95 (s, 3H, CH₃). ¹³C NMR (100 MHz, d₆-DMSO) δ 161.4 (C), 157.0 (CH), 149.4 (C), 144.1 (C), 126.4 (C), 39.7 (CH₃), 33.9 (CH₃).

### Synthesis of Compounds

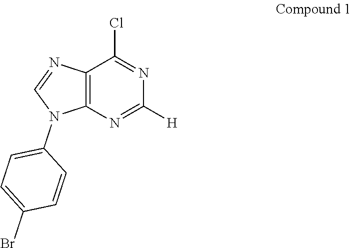 [SMILES: Clc1ncnc2c1ncn2-c1ccc(Br)cc1]

### Method 1

This compound was synthesised through general synthesis protocol I from 5-amino-4,6-dichloropyrimidine (100 mg, 0.61 mmol), 4-bromoaniline and (chloromethyl-ene)dimethyliminium chloride and was purified by chromatography on silica gel (eluent dichloromethane/methanol) to afford pure compound 1 (94%).

¹H NMR (400 MHz, CDCl₃) δ 8.82 (s, 1H, CH_{Ar}), 8.39 (s, 1H, CH_{Ar}), 8.77-8.72 (m, 2H, 2 CH_{Ar}), 7.66-7.61 (m, 2H, 2 CH_{Ar}); ¹³C NMR (100 MHz, CDCl₃) δ 152.9 (CH_{Ar}), 152.1 (C_{q}), 151.5 (C_{q}), 143.7 (CH_{Ar}), 133.4 (2 CH_{Ar}), 133.1 (C_{q}), 132.3 (C_{q}), 125.1 (2 CH_{Ar}), 123.0 (C_{q}); HRMS (ESI) calc. for C₁₁H₇BrClN₄: [M+H]⁺ 308.9537, found 308.9539.

### Method 2

To a solution of Intermediate Int-3 (50 mg, 0.26 mmol, 5 equivalents) in dioxane (375 μmoL), 4-bromoaniline (8 mg, 0.042 mmol) and benzenesulfonic acid (41 mg, 0.26 mmol) were added and the reaction mixture was heated in a microwave reactor at 100° C. for 30 min. The resulting mixture was allowed to reach r.t. and then CH₂Cl₂ (5 mL) and aqueous saturated NaHCO₃ (5 mL) were added. The aqueous layer was extracted 3 times with CH₂Cl₂ and the mixed resulting organic layers were then dried over magnesium sulfate and concentrated under reduced pressure. H NMR of the crude residue in CDCl₃ showed the complete disappearance of the starting aniline and the apparition of compound 1 (70%) and the corresponding 6-(4′-bromophenylamino)-9-(4″-bromophenyl)purine (30%).

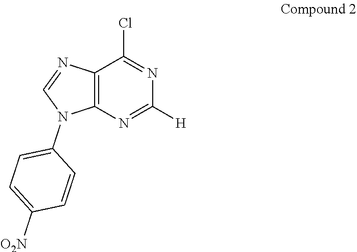 [SMILES: O=[N+]([O-])c1ccc(-n2cnc3c(Cl)ncnc32)cc1]

This compound was synthesised through general synthesis protocol I from 5-amino-4,6-dichloropyrimidine (100 mg, 0.61 mmol), 4-nitroaniline and (chloromethylene)di-methyliminium chloride, and was purified by chromatography on silica gel (eluent toluene/AcOEt) to afford pure compound 2 (63%).

¹H NMR (400 MHz, CDCl₃) δ 8.88 (s, 1H, CH_{Ar}), 8.52 (s, 1H, CH_{Ar}), 8.51-8.48 (m, 2H, 2 CH_{Ar}), 8.11-8.06 (m, 2H, 2 CH_{Ar}); ¹³C NMR (100 MHz, CDCl₃) δ 153.3 (CH_{Ar}), 152.5 (C_{q}), 151.4 (C_{q}), 147.3 (C_{q}), 143.0 (CH_{Ar}), 139.2 (C_{q}), 132.6 (C_{q}), 125.8 (2 CH_{Ar}), 123.4 (2 CH_{Ar}); HRMS (ESI) calc. for C11H7ClN₅O₂: [M+H]⁺ 276.0283, found 276.0282.

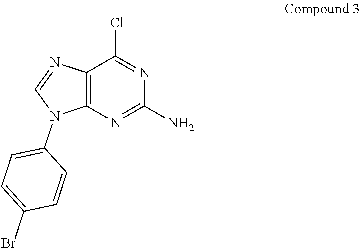 [SMILES: Nc1nc(Cl)c2ncn(-c3ccc(Br)cc3)c2n1]

This compound was synthesised through general synthesis protocol II from 2,5-amino-4,6-dichloropyrimidine (100 mg, 0.56 mmol), 4-bromoaniline and (chloromethyl-ene)dimethyliminium chloride, and was purified by chromatography on silica gel (eluent toluene/AcOEt) to afford pure compound 3 (83%).

¹H NMR (400 MHz, d₆-DMSO) δ 8.54 (s, 1H, CH_{Ar}), 7.85-7.75 (m, 4H, 4 CH), 7.07 (bs, 2H, NH₂); ¹³C NMR (100 MHz, d₆-DMSO) δ 160.2 (C_{q}), 153.5 (C_{q}), 150.0 (C_{q}), 141.6 (CH_{Ar}), 134.0 (C_{q}), 132.3 (2 CH_{Ar}), 125.2 (2 CH_{Ar}), 123.7 (C_{q}), 120.4 (C_{q}); HRMS (ESI) calc. for C₁₁H₈BrClN₅: [M+H]⁺ 323.9646, found 323.9646.

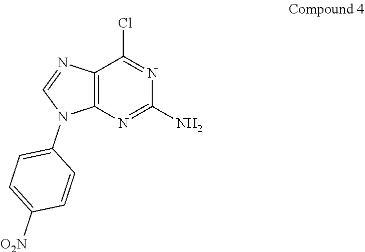 [SMILES: Nc1nc(Cl)c2ncn(-c3ccc([N+](=O)[O-])cc3)c2n1]

This compound was synthesised through general synthesis protocol II from 2,5-amino-4,6-dichloropyrimidine (100 mg, 0.56 mmol), 4-nitroaniline and (chloromethyl-ene)dimethyliminium chloride, and was purified by chromatography on silica gel (eluent toluene/AcOEt) to afford pure compound 4 (68%).

¹H NMR (400 MHz, d₆-DMSO) δ 8.72 (s, 1H, CH_{Ar}), 8.45-8.42 (m, 2H, 2 CH_{Ar}), 8.26-8.22 (m, 2H, 2 CH_{Ar}), 7.18 (bs, 2H, NH₂); ¹³C NMR (100 MHz, d₆-DMSO) δ 160.3 (C_{q}), 153.5 (C_{q}), 150.3 (C_{q}), 145.7 (C_{q}), 141.2 (CH_{Ar}), 140.1 (C_{q}), 124.9 (2 CH_{Ar}), 123.9 (C_{q}), 123.1 (2 CH_{Ar}); HRMS (ESI) calc. for C₁₁H₈ClN₆O₂: [M+H]⁺ 291.0392, found 291.0390.

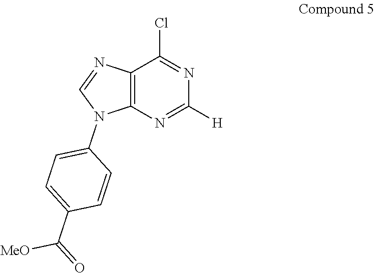 [SMILES: COC(=O)c1ccc(-n2cnc3c(Cl)ncnc32)cc1]

This compound was synthesised through general synthesis protocol I from 5-amino-4,6-dichloropyrimidine (100 mg, 0.61 mmol), methyl 4-aminobenzoate and (chloromethyl-ene)dimethyliminium chloride, and was purified by chromatography on silica gel (cyclohexane/AcOEt) to afford pure compound 5 (80%).

¹H NMR (400 MHz, CDCl₃) δ 8.85 (s, 1H, CH_{Ar}), 8.48 (s, 1H, CH_{Ar}), 8.32-8.26 (m, 2H, 2 CH_{Ar}), 7.91-7.86 (m, 2H, 2 CH_{Ar}), 3.99 (s, 3H, CH₃); ¹³C NMR (100 MHz, CDCl₃) δ 165.9 (CO) 153.0 (CH_{Ar}), 152.2 (C_{q}), 151.5 (C_{q}), 143.6 (CH_{Ar}), 137.8 (C_{q}), 132.5 (C_{q}), 131.7 (2 CH_{Ar}), 130.5 (C_{q}), 123.0 (2 CH_{Ar}), 52.7 (CH₃); HRMS (ESI) calc. for C₁₃H₁₀ClN₄O₂: [M+H]⁺ 289.0487, found 289.0489.

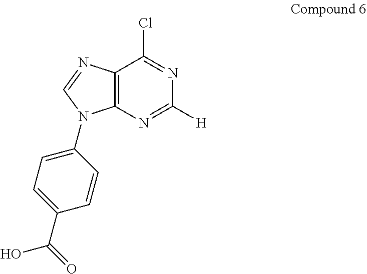 [SMILES: O=C(O)c1ccc(-n2cnc3c(Cl)ncnc32)cc1]

To a solution of 5-amino-4,6-dichloropyrimidine (100 mg, 0.61 mmol) in anhydrous dioxane (2 mL/mmol) under argon atmosphere, were successively added methyl 4-aminobenzoic acid (84 mg, 0.61 mmol) and APTS.H₂O (58 mg, 0.305 mmol). The solution was stirred at reflux for 18 h. After cooling to room temperature, the mixture was filtered and the resulting solid was washed with dioxane. 4.5 mL of a 0.2 M solution of (chloromethylene)dimethyliminium chloride (115 mg, 0.9 mmol) in anhydrous DMF was then added dropwise to the solid under argon atmosphere and the resulting mixture was stirred for 30 min. Water (300 μL) was added and the resulted mixture was concentrated under vacuo. The resulting solid was washed with water to give 6 as a white solid (72%).

¹H NMR (400 MHz, d₆-DMSO) δ 13.26 (bs, 1H CO₂H) 9.22 (s, 1H, CH_{Ar}), 8.89 (s, 1H, CH_{Ar}), 8.22-8.14 (m, 2H, 2 CH_{Ar}), 8.14-8.07 (m, 2H, 2 CH_{Ar}); ¹³C NMR (100 MHz, d₆-DMSO) δ 166.5 (CO) 152.3 (CH_{Ar}), 151.4 (C_{q}), 149.8 (C_{q}), 146.1 (CH_{Ar}), 137.6 (C_{q}), 131.7 (C_{q}), 130.7 (2 CH_{Ar}), 130.3 (C_{q}), 123.1 (2 CH_{Ar}); HRMS (ESI) calc. for C₁₂H₈ClN₄O₂: [M+H]⁺ 275.0330, found 275.0326.

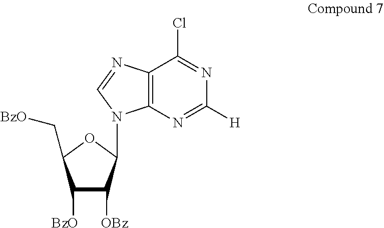

### Method 1

This compound was synthesised through general synthesis protocol V from Intermediate Int-1 and was purified by chromatography on silica gel (cyclohexane/AcOEt) to afford pure compound 7 (50%).

For characterization, see: V. Yadav, C. K. Chu, R. H. Rais, O. N. Al Safarjalani, V. Guarcello, F. N. M. Naguib, M. H. el Kouni, J. Med. Chem., 2004, 1987-1996.

### Method 2

### Preparation of Solution A:

To a stirred solution of 1-O-Acetyl-2,3,5-tri-O-benzoyl-beta-D-ribofuranose (100 mg, 0.2 mmol) in anhydrous CH₂Cl₂ (3.3 mL) under argon atmosphere was added a 1 M solution of TiCl₄ in anhydrous CH₂Cl₂ (180 μL). The resulting mixture was stirred 1 h 30 and then diluted with water (3 mL). The organic phase was then dried over magnesium sulfate and concentrated. Anhydrous acetonitrile (1 mL) was added to the residue to give solution A.

### Preparation of Solution B:

To a solution of Intermediate Int-1 (20 mg, 0.1 mmol) in dry acetonitrile (1 mL) under argon atmosphere was added hexamethyldisilazane (HMDS, 42 μL, 0.2 mmol) and trimethylsilyl chloride (TMSCl, 38 μL, 0.3 mmol). The resulting mixture was stirred at reflux for 2 h and then concentrated. Anhydrous acetonitrile (1 mL) was added to the residue to give solution B.

### Final Reaction:

Solution A was added dropwise to solution B and the resulting mixture was stirred under reflux for 16 h and then allowed to reach r.t. The solution was diluted with CH₂Cl₂ (15 mL) and washed with saturated aqueous NaHCO₃ (15 mL). The aqueous layer was extracted 3 times with CH₂Cl₂ and the resulting mixed organic layers were then dried over magnesium sulfate. After concentration under reduced pressure, the residue was purified by chromatography on silica gel (eluent cyclohexane/ethyl acetate) to afford pure compound 7 (34%).

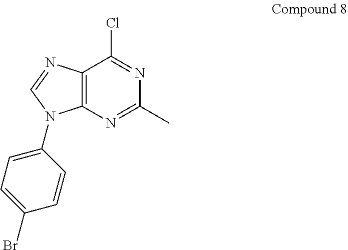 [SMILES: Cc1nc(Cl)c2ncn(-c3ccc(Br)cc3)c2n1]

This compound was synthesised through general synthesis protocol I from 5-amino-4,6-dichloro-2-methylpyrimidine (100 mg, 0.56 mmol), 4-bromoaniline and (chloromethyl-ene)dimethyliminium chloride, and was purified by chromatography on silica gel (eluent cyclohexane/ethyl acetate) to afford pure compound 8 (80%).

¹H NMR (400 MHz, CDCl₃) δ 8.29 (s, 1H, CHₐᵣ), 7.75-7.71 (m, 2H, 2 CHₐᵣ), 7.65-7.60 (m, 2H, 2 CHₐᵣ), 2.88 (s, 3H, CH₃). ¹³C NMR (100 MHz, CDCl₃) δ 163.7 (C_{Q}), 152.0 (C_{Q}), 151.4 (C_{Q}), 143.1 (CHₐᵣ), 133.3 (2 CHₐᵣ), 133.2 (C_{Q}), 130.0 (C_{Q}), 125.1 (2 CHₐᵣ), 122.7 (C_{Q}), 26.0 (CH₃). HRMS (ESI) calc. for C₁₂H₉BrClN₄: [M+H]⁺ 322.9694, found 322.9693.

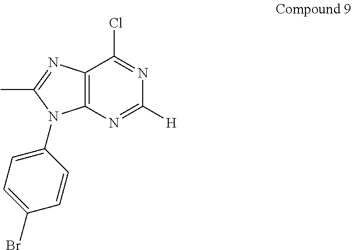 [SMILES: Cc1nc2c(Cl)ncnc2n1-c1ccc(Br)cc1]

### Method 1

This compound was synthesised through general synthesis protocol I from 5-amino-4,6-dichloropyrimidine (100 mg, 0.61 mmol), 4-bromoaniline and N,N-dimethylacetamide, and was purified by chromatography on silica gel (eluent cyclohexane/AcOEt) to afford pure compound 9 (73%).

¹H NMR (400 MHz, CDCl₃) δ 8.67 (s, 1H, CHₐᵣ), 7.78-7.74 (m, 2H, 2 CHₐᵣ), 7.33-7.26 (m, 2H, 2 CHₐᵣ), 2.62 (s, 3H, CH₃). ¹³C NMR (100 MHz, CDCl₃) δ 155.0 (C_{Q}), 153.8 (C_{Q}), 152.2 (CHₐᵣ), 149.9 (C_{Q}), 133.7 (2 CHₐᵣ), 132.6 (C_{Q}), 131.3 (C_{Q}), 128.9 (2 CHₐᵣ), 124.5 (C_{Q}), 15.3 (CH₃). HRMS (ESI) calc. for C₁₂H₉BrClN₄: [M+H]⁺ 322.9694, found 322.9695.

### Method 2

### Preparation of Solution A:

To a solution of 5-amino-4,6-dichloropyrimidine (100 mg, 0.61 mmol) in anhydrous dioxane (1.22 mL) under argon atmosphere, were successively added 4-bromoaniline (105 mg, 0.61 mmol) and APTS.H₂O (58 mg, 0.305 mmol). The solution was stirred at reflux for 18 h and then concentrated under reduced pressure. The residue was diluted in AcOEt (5 mL) and washed with a saturated aqueous solution of NaHCO₃. The aqueous layer was extracted 3 times with ethyl acetate and the resulting organic layer was then dried over magnesium sulfate and concentrated under reduced pressure. The crude product was solubilized in anhydrous 1,2-dimethoxyethane (6.1 mL) to give solution A.

### Preparation of Solution B:

To a solution N,N-dimethylacetamide (0.113 mL, 1.22 mmol) in dry 1,2-dichloroethane (4.88 mL) under argon atmosphere, was added oxalyl chloride (0.103 mL, 1.22 mmol) and the resulting mixture was stirred at 35° C. for 6 h to give solution B.

### Final Reactions:

Solution A was then added drop wise to solution B at 35° C. The resulting mixture was stirred 2 h at 35° C. followed by 2 h at reflux and then diluted with CH₂Cl₂ (50 mL). The resulting solution was washed with saturated aqueous NaHCO₃. The aqueous layer was extracted 3 times with CH₂Cl₂ and the resulting mixed organic layers were then dried over magnesium sulfate and concentrated under reduced pressure. The residue was purified by chromatography on silica gel (cyclohexane/AcOEt) to afford pure compound 9 (74%).

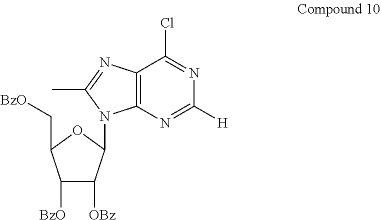

This compound was synthesised through general synthesis protocol V from Intermediate Int-3 (20 mg, 0.094 mmol) and was purified by chromatography on silica gel (cyclohexane/AcOEt) to afford pure compound 10 (47%).

¹H NMR (400 MHz, CDCl₃) δ 8.53 (s, 1H, H−2), 8.05-7.90 (m, 6H, 6 CHₐᵣ), 7.62-7.52 (m, 3H, 3 CHₐᵣ), 7.45-7.33 (m, 6H, 6 CHₐᵣ), 6.56 (dd, 1H, J_{1′,2′}=3.4 Hz, J_{2′,3′}=6.1 Hz, H−2′), 6.45 (dd, 1H, J_{3′,4′}=6.9 Hz, H−3′), 6.20 (d, 1H, H−1′), 4.92 (dd, 1H, J_{4′,5′a}=3.3 Hz, J_{5′a,5′b}=12.2 Hz, H−5′a), 4.81 (ddd, 1H, J_{4′,5′b}=4.6 Hz, H−4′), 4.65 (dd, 1H, H−5′b), 2.77 (3H, CH₃). ¹³C NMR (100 MHz, CDCl₃) δ 166.1 (C), 165.5 (C), 165.4 (C), 155.1 (C), 152.3 (C), 151.4 (CH), 149.6 (C), 134.0 (CH), 133.8 (CH), 133.4 (CH), 131.3 (C), 129.9-129.6 (CH), 129.4 (C), 128.8 (C), 128.7-128.3 (CH), 128.5 (C), 88.1 (CH), 80.2 (CH), 73.8 (CH), 71.0 (CH), 62.8 (CH₂), 14.8 (CH₃). HRMS (ESI) calc. for C₃₂H₂₆O₇ClN₄: [M+H]⁺ 613.1485, found 613.1483.

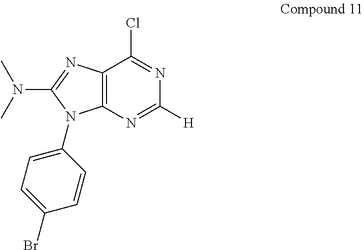 [SMILES: CN(C)c1nc2c(Cl)ncnc2n1-c1ccc(Br)cc1]

This compound was synthesised through general synthesis protocol IV from 5-amino-4,6-dichloropyrimidine (100 mg, 0.61 mmol), 4-bromoaniline and (dichloromethyl-ene)dimethyliminium chloride and was purified by chromatography on silica gel (cyclohexane/AcOEt) to afford pure compound 11 (77%).

¹H NMR (400 MHz, CDCl₃) δ 8.43 (s, 1H, CHₐᵣ), 7.74-7.69 (m, 2H, 2 CHₐᵣ), 7.41-7.36 (m, 2H, 2 CHₐᵣ), 2.97 (s, 6H, 2 CH₃). ¹³C NMR (100 MHz, CDCl₃) δ 157.6 (C), 154.7 (C), 149.5 (CH), 144.3 (C), 134.2 (C), 133.4 (2 CH), 131.4 (C), 128.3 (2 CH), 123.4 (C), 40.9 (2 CH₃). HRMS (ESI) calc. for C₁₃H₁₂BrClN₅: [M+H]⁺ 351.9959, found 351.9957.

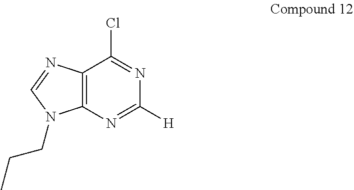

This compound was synthesised through general synthesis protocol III from 5-amino-4,6-dichloropyrimidine (100 mg, 0.61 mmol), 3-propylamine and (chloromethyl-ene)dimethyliminium chloride and was purified by chromatography on silica gel (cyclohexane/AcOEt) to afford pure compound 12 (68%).

¹H NMR (400 MHz, CDCl₃) δ 8.75 (s, 1H, CHₐᵣ), 8.12 (s, 1H, CHₐᵣ), 4.27 (t, 2H, J=7.3 Hz, NCH₂), 1.96 (st, 2H, J=7.3 Hz, CH₂CH₃), 0.98 (t, 3H, CH₃). ¹³C NMR (100 MHz, CDCl₃) δ 152.0 (Cq, CH), 151.2 (C), 145.3 (CH), 131.8 (C), 46.3 (CH₂), 23.4 (CH₂), 11.3 (CH₃). HRMS (ESI) calc. for C₈H₁₀ClN₄: [M+H]⁺ 197.0589 found 197.0589.

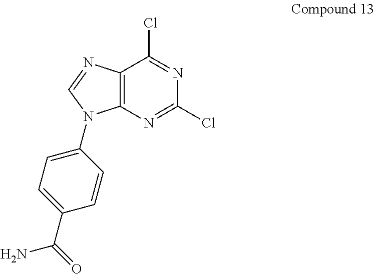 [SMILES: NC(=O)c1ccc(-n2cnc3c(Cl)nc(Cl)nc32)cc1]

A solution of pyrimidine Intermediate Int-4 (48 mg, 0.19 mmol), 4-aminobenzamide (22 mg, 0.16 mmol) and benzenesulfonic acid (25 mg, 0.16 mmol) in anhydrous DMF (2 mL) was stirred at room temperature under argon atmosphere for 3 days. The solution was then diluted with ethyl acetate, washed with saturated aqueous NaHCO₃, dried over MgSO₄ and concentrated. The residue was purified by chromatography on silica gel (eluent: CH₂Cl₂/MeOH, 10/0 to 9/1) to give compound 13 as a white solid (43 mg, 86%).

¹H NMR (400 MHz, d₆-DMSO) δ 9.19 (s, 1H), 8.16-8.10 (m, 2H+NH), 7.98-7.93 (m, 2H), 7.56 (1H, NH). ¹³C NMR (100 MHz, DMSO) δ 166.8 (CONH₂), 152.9 (C_{IV}), 151.7 (C_{IV}), 150.2 (C_{IV}), 147.1 (CH_{Ar}), 135.8 (C_{IV}), 134.3 (C_{IV}), 131.3 (C_{IV}), 128.9 (CH_{Ar}), 123.3 (CH_{Ar}). HRMS (ESI) calc. for C₁₂H₈Cl₂N₅O: [M+H]+ 308.01001, found 308.01004.

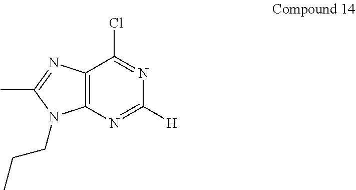

A solution of 5-amino-4-chloro-6-(propylamino)pyrimidine (100 mg, 0.54 mmol) in N,N-dimethylacetamide dimethyl acetal (450 μL, 10% methanol) under argon atmosphere is stirred at 60° C. for 2 h. The solution was diluted in DCM and washed with a saturated aqueous solution of NaHCO₃. The aqueous layer was extracted 3 times with DCM and the resulting organic layer was then dried over MgSO₄ and concentrated in vacuo. The crude product was diluted in anhydrous DMF (5.4 mL) under argon atmosphere and APTS.H₂O (0.27 mmol, 52 mg) was added. The resulting mixture was stirred at 75° C. for 1 h. The solution was diluted in DCM and washed with a saturated aqueous solution of NaHCO₃. The aqueous layer was extracted 3 times with DCM and the resulting organic layer was then dried over magnesium sulfate and concentrated in vacuo. The residue was purified by chromatography on silica gel (cyclohexane/AcOEt) to afford pure compound 15 (55%).

¹H NMR (400 MHz, CDCl₃) δ 8.67 (s, 1H, CHₐᵣ), 4.20 (t, 2H, J=7.3 Hz, CH₂N), 2.70 (s, 3H, CH₃), 1.93-1.82 (m, 2H, CH₂CH₂N), 0.98 (t, 3H, J=7.4 Hz, CH₃CH₂). ¹³C NMR (100 MHz, CDCl₃) δ 155.1 (C), 153.3 (C), 151.2 (CH), 149.0 (C), 131.1 (C), 45.1 (CH₂), 23.1 (CH₂), 14.7 (CH₃), 11.3 (CH₃). HRMS (ESI) calc. for C₉H₁₂ClN₄: [M+H]⁺, 211.0745 found 211.0744.

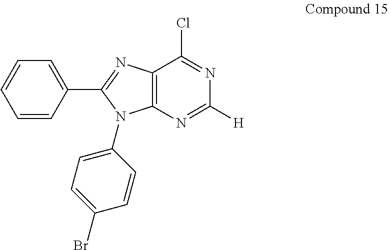 [SMILES: Clc1ncnc2c1nc(-c1ccccc1)n2-c1ccc(Br)cc1]

### Preparation of Solution A:

To a solution of 5-amino-4,6-dichloropyrimidine (100 mg, 0.61 mmol) in anhydrous dioxane (1.22 mL) under argon atmosphere, were successively added 4-bromoaniline (105 mg, 0.61 mmol) and APTS.H₂O (58 mg, 0.305 mmol). The solution was stirred at reflux for 18 h and then concentrated under reduced pressure. The residue was diluted in AcOEt (5 mL) and washed with a saturated aqueous solution of NaHCO₃. The aqueous layer was extracted 3 times with ethyl acetate and the resulting organic layer was then dried over magnesium sulfate and concentrated under reduced pressure. The crude product was suspended in anhydrous 1,2-dimethoxyethane (6.1 mL) to give solution A.

### Preparation of Solution B:

To a solution N,N-dimethylbenzamide (0.182 g, 1.22 mmol) in dry 1,2-dichloroethane (1.22 mL) under argon atmosphere, was added oxalyl chloride (0.103 mL, 1.22 mmol) and the resulting mixture was stirred at 35° C. for 6 h to give solution B.

Solution A was then added drop by drop to solution B at 35° C. The resulting mixture was stirred 2 h at 35° C. followed by 2 h at reflux and then diluted with CH₂Cl₂ (50 mL) and washed with a saturated aqueous solution of NaHCO₃. The aqueous layer was extracted 3 times with CH₂Cl₂ and the resulting organic layer was then dried over magnesium sulfate and concentrated under reduced pressure. The residue was purified by chromatography on silica gel (cyclohexane/AcOEt) to afford pure compound 16 (78%).

¹H NMR (400 MHz, CDCl₃) δ 8.72 (s, 1H, CHₐᵣ), 7.71-7.57 (m, 4H, 4 CHₐᵣ), 7.52-7.43 (m, 1H, CHₐᵣ), 7.43-7.34 (m, 2H, 2 CHₐᵣ), 7.30-7.17 (m, 2H, 2 CHₐᵣ). ¹³C NMR (100 MHz, CDCl₃) δ 154.9 (C), 154.1 (C), 152.4 (CH), 150.8 (C), 133.5 (C), 133.3 (2 CH), 131.8 (C), 131.3 (CH), 129.9 (2 CH), 128.9 (4 CH), 128.1 (C), 123.8 (C). HRMS (ESI) calc. for C₁₇H₁₁BrClN₄: [M+H]⁺, 384.9850 found 384.9853.

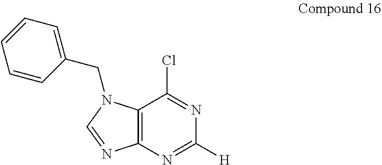 [SMILES: Clc1ncnc2ncn(Cc3ccccc3)c12]

To a solution of N⁵-benzyl-6-chloropyrimidine-4,5-diamine (20 mg, 0.085 mmol, synthesized according to J. Liu, Q. Dang, Z. Wei, F. Shi, X. Bai, J. Comb. Chem, 2006, 8, 410-416) in anhydrous DMF (850 μL) under argon atmosphere at room temperature, was added (chloromethylene)dimethyliminium chloride (16 mg, 0.128 mmol). The resulting solution was stirred for 30 min and then diluted with CH₂Cl₂ (5 mL) and washed with a saturated aqueous solution of NaHCO₃. The aqueous layer was extracted 3 times with CH₂Cl₂ and the resulting organic layer was then dried over magnesium sulfate and concentrated under reduced pressure to afford pure compound 17 (100%).

¹H NMR (400 MHz, CDCl₃) δ 8.90 (s, 1H, CHₐᵣ), 8.23 (s, 1H, CHₐᵣ), 7.44-7.35 (m, 3H, 3 CHₐᵣ), 7.21-7.13 (m, 2H, 2 CHₐᵣ), 5.69 (s, 2H, CH₂). ¹³C NMR (100 MHz, CDCl₃) δ 162.2 (C), 152.8 (CH), 149.2 (CH), 143.4 (C), 134.7 (C), 129.6 (2 CH), 129.1 (CH), 127.2 (2 CH), 122.7 (C), 50.9 (CH₂).

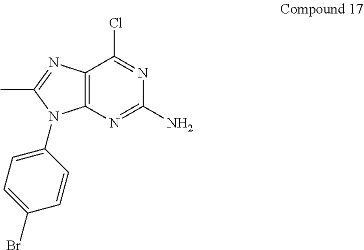 [SMILES: Cc1nc2c(Cl)nc(N)nc2n1-c1ccc(Br)cc1]

This compound was synthesised through general synthesis protocol II from 2,5-amino-4,6-dichloropyrimidine (100 mg, 0.56 mmol), 4-bromoaniline and a 0.2 M solution of oxalyl chloride in N,N-dimethylacetamide (8.5 mL), and was purified by chromatography on silica gel (eluent toluene/AcOEt) to afford pure compound 17 (26%).

¹H NMR (400 MHz, DMSO-d₆) δ 7.83-7.78 (m, 2H, 2 CHₐᵣ), 7.53-7.48 (m, 2H, 2 CHₐᵣ), 6.84 (bs, 2H, NH₂), 2.33 (s, 3H, CH₃). ¹³C NMR (400 MHz, DMSO-d₆) δ 159.8 (C), 155.9 (C), 150.6 (C), 147.9 (C), 133.4 (C), 132.6 (2 CHₐᵣ), 129.9 (2 CHₐᵣ), 122.4 (C), 122.3 (C), 14.4 (CH₃). HRMS (ESI) calc. for C₁₂H₁₀BrClN₅: [M+H]⁺ 337.9803, found 337.9799.

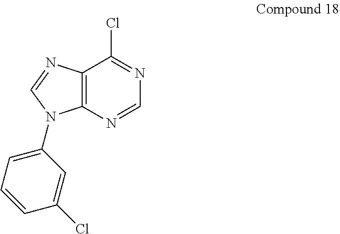 [SMILES: Clc1cccc(-n2cnc3c(Cl)ncnc32)c1]

This compound was synthesised through general synthesis protocol I from 5-amino-4,6-dichloropyrimidine (100 mg, 0.61 mmol), 3-chloroaniline and (chloromethylene)dimethyliminium chloride, and was purified by chromatography on silica gel (cyclohexane/AcOEt) to afford pure compound 18 (84%).

¹H NMR (400 MHz, CDCl₃) δ 8.84 (s, 1H, CHₐᵣ), 8.41 (s, 1H, CHₐᵣ), 7.80 (t, 1H, J=2.0 Hz, CHₐᵣ), 7.68-7.63 (m, 1H, CHₐᵣ), 7.56 (t, 1H, J=8.0 Hz, CHₐᵣ), 7.52-7.49 (m, 1H, CHₐᵣ). ¹³C NMR (400 MHz, CDCl₃) δ 153.0 (CHₐᵣ), 152.1 (C), 151.5 (C), 143.7 (CHₐᵣ), 136.0 (C), 135.1 (C), 132.3 (C), 131.3 (CHₐᵣ), 129.3 (CHₐᵣ), 123.9 (CHₐᵣ), 121.6 (CHₐᵣ). HRMS (ESI) calc. for C₁₁H₇Cl₂N₄: [M+H]⁺ 265.0042, found 265.0042.

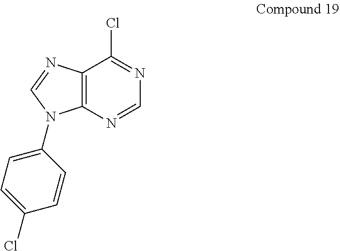 [SMILES: Clc1ccc(-n2cnc3c(Cl)ncnc32)cc1]

This compound was synthesised through general synthesis protocol I from 5-amino-4,6-dichloropyrimidine (100 mg, 0.61 mmol), 4-chloroaniline and (chloromethylene)dimethyliminium chloride, and was purified by chromatography on silica gel (cyclohexane/AcOEt) to afford pure compound 19 (86%).

¹H NMR (400 MHz, CDCl₃) δ 8.83 (s, 1H, CHₐᵣ), 8.39 (s, 1H, CHₐᵣ), 7.73-7.66 (m, 2H, 2 CHₐᵣ), 7.62-7.57 (m, 2H, 2 CHₐᵣ). ¹³C NMR (400 MHz, CDCl₃) δ 152.9 (CHₐᵣ), 152.1 (C), 151.5 (C), 143.8 (CHₐᵣ), 135.1 (C), 132.5 (C), 132.3 (C), 130.5 (2 CH), 124.9 (2 CHₐᵣ). HRMS (ESI) calc. for C₁₁H₇Cl₂N₄: [M+H]⁺ 265.0042, found 265.0039.

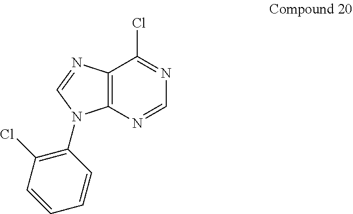 [SMILES: Clc1ccccc1-n1cnc2c(Cl)ncnc21]

This compound was synthesised through general synthesis protocol I from 5-amino-4,6-dichloropyrimidine (100 mg, 0.61 mmol), 2-chloroaniline and (chloromethylene)dimethyliminium chloride, and was purified by chromatography on silica gel (cyclohexane/AcOEt) to afford pure compound 20 (22%).

¹H NMR (400 MHz, CDCl₃) δ 8.78 (s, 1H, CHₐᵣ), 8.30 (s, 1H, CHₐᵣ), 7.71-7.63 (m, 1H, CHₐᵣ), 7.59-7.50 (m, 3H, 3 CHₐᵣ). ¹³C NMR (400 MHz, CDCl₃) δ 153.0 (CHₐᵣ), 152.3 (C), 151.9 (C), 145.4 (CHₐᵣ), 131.6 (CHₐᵣ), 131.5 (C), 131.4 (C), 131.2 (CHₐᵣ), 131.1 (C), 129.1 (CHₐᵣ), 128.4 (CHₐᵣ). HRMS (ESI) calc. for C₁₁H₇Cl₂N₄: [M+H]⁺ 265.0042, found 265.0042.

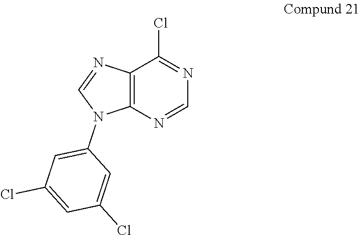 [SMILES: Clc1cc(Cl)cc(-n2cnc3c(Cl)ncnc32)c1]

This compound was synthesised through general synthesis protocol I from 5-amino-4,6-dichloropyrimidine (100 mg, 0.61 mmol), 3,5-dichloroaniline and (chloromethylene)dimethyliminium chloride, and was purified by chromatography on silica gel (cyclohexane/AcOEt) to afford pure compound 21 (80%).

¹H NMR (400 MHz, CDCl₃) δ 8.86 (s, 1H, CHₐᵣ), 8.41 (s, 1H, CHₐᵣ), 7.74 (d, 2H, J=1.8 Hz, 2 CHₐᵣ), 7.51 (t, 1H, J=1.8 Hz, CHₐᵣ). ¹³C NMR (400 MHz, CDCl₃) δ 153.2 (CHₐᵣ), 152.4 (C), 151.4 (C), 143.3 (CHₐᵣ), 136.8 (2 C), 135.8 (C), 132.4 (C), 129.2 (CHₐᵣ), 121.9 (2 CHₐᵣ). HRMS (ESI) calc. for C₁₁H₆Cl₃N₄: [M+H]⁺ 298.9653, found 298.9650.

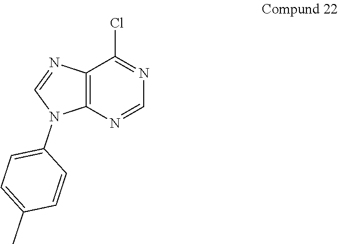 [SMILES: Cc1ccc(-n2cnc3c(Cl)ncnc32)cc1]

This compound was synthesised through general synthesis protocol I from 5-amino-4,6-dichloropyrimidine (100 mg, 0.61 mmol), 4-methylaniline and (chloromethylene)dimethyliminium chloride, and was purified by chromatography on silica gel (cyclohexane/AcOEt) to afford pure compound 22 (85%).

¹H NMR (400 MHz, CDCl₃) δ 8.81 (s, 1H, CHₐᵣ), 8.37 (s, 1H, CHₐᵣ), 7.60-7.52 (m, 2H, 2 CHₐᵣ), 7.45-7.35 (m, 2H, 2 CHₐᵣ), 2.46 (s, 3H, CH₃). ¹³C NMR (400 MHz, CDCl₃) δ 158.8 (CHₐᵣ), 151.8 (C), 151.7 (C), 144.4 (CHₐᵣ), 139.5 (C), 132.2 (C), 131.5 (C), 130.8 (2 CHₐᵣ), 123.7 (2 CHₐᵣ), 21.3 (CH₃). HRMS (ESI) calc. for C₁₂H₁₀ClN₄: [M+H]⁺ 245.0589, found 245.0590.

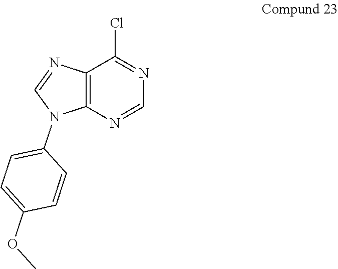 [SMILES: COc1ccc(-n2cnc3c(Cl)ncnc32)cc1]

This compound was synthesised through general synthesis protocol I from 5-amino-4,6-dichloropyrimidine (100 mg, 0.61 mmol), 4-methoxyaniline and (chloromethylene)dimethyliminium chloride, and was purified by chromatography on silica gel (cyclohexane/AcOEt) to afford pure compound 23 (83%).

¹H NMR (400 MHz, CDCl₃) δ 8.80 (s, 1H, CHₐᵣ), 8.33 (s, 1H, CHₐᵣ), 7.60-7.55 (m, 2H, 2 CHₐᵣ), 7.14-7.08 (m, 2H, 2 CHₐᵣ), 3.89 (s, 3H, CH₃). ¹³C NMR (400 MHz, CDCl₃) δ 159.8 (C), 152.4 (CHₐᵣ), 151.5 (C), 151.4 (C), 144.3 (CHₐᵣ), 131.7 (C), 126.4 (C), 125.2 (2 CHₐᵣ), 115.0 (2 CHₐᵣ), 55.5 (CH₃). HRMS (ESI) calc. for C₁₂H₁₀ClN₄O: [M+H]⁺ 261.0538, found 261.0536.

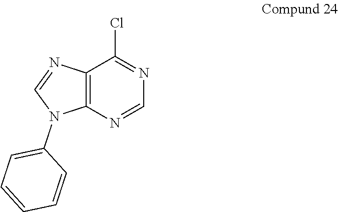 [SMILES: Clc1ncnc2c1ncn2-c1ccccc1]

This compound was synthesised through general synthesis protocol I from 5-amino-4,6-dichloropyrimidine (100 mg, 0.61 mmol), aniline and (chloromethylene)dimethyliminium chloride, and was purified by chromatography on silica gel (CH₂Cl₂/MeOH) to afford pure compound 24 (81%).

¹H NMR (400 MHz, CDCl₃) δ 8.80 (s, 1H, CHₐᵣ), 8.41 (s, 1H, CHₐᵣ), 7.69-7.72 (m, 2H, 2 CHₐᵣ), 7.58-7.62 (m, 2H, 2 CHₐᵣ), 7.49-7.53 (m, 1H, CHₐᵣ).

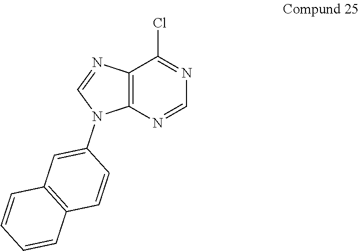 [SMILES: Clc1ncnc2c1ncn2-c1ccc2ccccc2c1]

This compound was synthesised through general synthesis protocol I from 5-amino-4,6-dichloropyrimidine (100 mg, 0.61 mmol), 2-naphthylamine and (chloromethylene)dimethyliminium chloride, and was purified by chromatography on silica gel (CH₂Cl₂/MeOH) to afford pure compound 25 (86%).

¹H NMR (400 MHz, CDCl₃) δ 8.85 (s, 1H, CHₐᵣ), 8.52 (s, 1H, CHₐᵣ), 8.19 (d, 1H, J=2.1 Hz, CHₐᵣ), 8.08 (d, 1H, J=8.8 Hz, CHₐᵣ), 7.94-7.97 (m, 2H, 2 CHₐᵣ), 7.81 (dd, 1H, J=8.8 Hz, 2.1 Hz, CHₐᵣ), 7.59-7.64 (m, 2H, 2 CHₐᵣ).

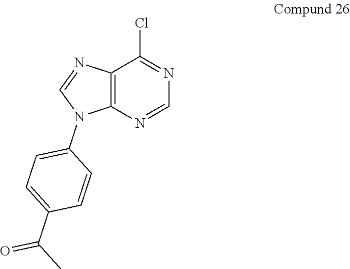 [SMILES: CC(=O)c1ccc(-n2cnc3c(Cl)ncnc32)cc1]

This compound was synthesised through general synthesis protocol I from 5-amino-4,6-dichloropyrimidine (100 mg, 0.61 mmol), 4′-aminoacetophenone and (chloromethylene)dimethyliminium chloride, and was purified by chromatography on silica gel (CH₂Cl₂/MeOH) to afford pure compound 26 (54%).

¹H NMR (400 MHz, CDCl₃) δ 8.84 (s, 1H, CHₐᵣ), 8.50 (s, 1H, CHₐᵣ), 8.18-8.21 (m, 2H, 2 CHₐᵣ), 7.91-7.94 (m, 2H, 2 CHₐᵣ), 2.68 (s, 3H, CH₃).

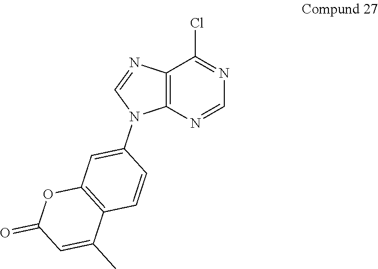 [SMILES: Cc1cc(=O)oc2cc(-n3cnc4c(Cl)ncnc43)ccc12]

This compound was synthesised through general synthesis protocol I from 5-amino-4,6-dichloropyrimidine (100 mg, 0.61 mmol), 7-amino-4-methylcoumarin and (chloromethylene)dimethyliminium chloride, and was purified by chromatography on silica gel (CH₂Cl₂/MeOH) to afford pure compound 27 (58%).

¹H NMR (400 MHz, CDCl₃) δ 8.87 (s, 1H, CHₐᵣ), 8.51 (s, 1H, CHₐᵣ), 7.87 (d, 1H, J=2.1 Hz, CHₐᵣ), 7.85 (d, 1H, J=8.5 Hz, CHₐᵣ), 7.78 (dd, 1H, J=8.5 Hz, 2.1 Hz, CHₐᵣ), 6.41 (d, 1H, J=1.2 Hz, CHₐᵣ), 2.53 (d, 3H, J=1.2 Hz, CH₃).

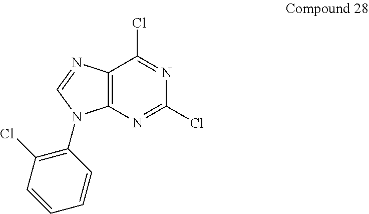 [SMILES: Clc1nc(Cl)c2ncn(-c3ccccc3Cl)c2n1]

A suspension of Intermediate Int-4 (100 mg, 0.40 mmol), 2-chloroaniline (49 μL, 0.47 mmol) and benzenesulfonic acid (62 mg, 0.40 mmol) in anhydrous DMF (4 mL) was heated at 50° C. under argon atmosphere for 24 h. Additional 2-chloroaniline (49 μL, 0.47 mmol) was added and the mixture was further heated at 50° C. for 24 h. The solution was then diluted with ethyl acetate, washed with saturated aqueous NaHCO₃, dried over MgSO₄ and concentrated. The residue was purified by two successive chromatography on silica gel (eluent: cyclohexane/ethyl acetate 8/2 then CH₂Cl₂/MeOH, 98/2) to give compound 28 as a white solid (67 mg, 56%). ¹H NMR (400 MHz, d₆-DMSO) δ 9.01 (s, 1H), 7.89-7.74 (m, 2H), 7.73-7.59 (m, 2H)³C NMR (100 MHz, d₆-DMSO) δ 153.8 (C), 151.9 (C), 150.3 (C), 148.3 (CH), 132.0 (CH), 130.5 (C), 130.4 (CH), 130.4 (C), 130.3 (C), 130.0 (CH), 128.6 (CH).

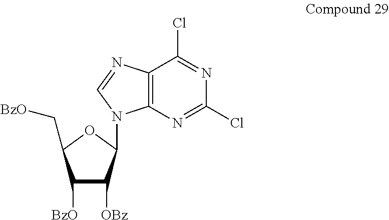

This compound was synthesised through general synthesis protocol V from Intermediate Int-5 and was purified by chromatography on silica gel (cyclohexane/AcOEt) to afford pure compound 29 (37%). Unreacted Intermediate Int-5 was recovered (55%).

¹H NMR (400 MHz, CDCl₃) δ 8.28 (s, 1H, H−8), 8.05-7.90 (m, 6H, 6 CHₐᵣ), 7.62-7.52 (m, 3H, 3 CHₐᵣ), 7.45-7.33 (m, 6H, 6 CHₐᵣ), 6.48 (d, 1H, J_{1′,2′}=5.5 Hz, H−1′), 6.18 (dd, 1H, J_{2′,3′}=5.7 Hz, H−2′), 6.13 (dd, 1H, J_{3′,4′}=4.2 Hz, H−3′), 4.92 (dd, 1H, J_{4′,5′a}=3.2 Hz, J_{5′a,5′b}=12.2 Hz, H−5′a), 4.87 (ddd, 1H, J_{4′,5′b}=4.1 Hz, H−4′), 4.73 (dd, 1H, H−5′b).

For characterization, see: B. Fraser-Reid, P. Ganney, C. V. S. Ramamurty, A. M. Gomez, J. Cristobal Lopez, Chem. Commun., 2013, 3251.

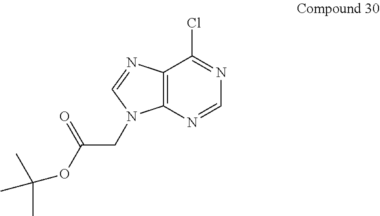 [SMILES: CC(C)(C)OC(=O)Cn1cnc2c(Cl)ncnc21]

This compound was synthesised through general synthesis protocol III from 5-amino-4,6-dichloropyrimidine (100 mg, 0.61 mmol), glycine tert-butyl ester hydrochloride and (chloromethylene)dimethyliminium chloride and was purified by chromatography on silica gel (cyclohexane/AcOEt) to afford pure compound 30 (38%).

¹H NMR (400 MHz, CDCl₃) δ 8.72 (s, 1H, CHₐᵣ), 8.19 (s, 1H, CHₐᵣ), 4.94 (s, 2H, CH₂), 1.45 (s, 9H, 3 CH₃).

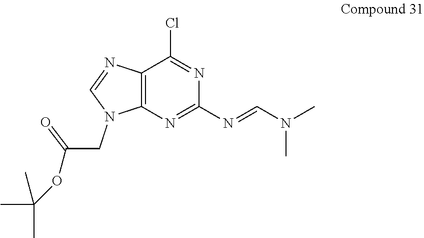 [SMILES: CN(C)/C=N/c1nc(Cl)c2ncn(CC(=O)OC(C)(C)C)c2n1]

This compound was synthesised through general synthesis protocol III from 2,5-amino-4,6-dichloropyrimidine (100 mg, 0.56 mmol), glycine tert-butyl ester hydrochloride (3 eq) and (chloromethylene)dimethyliminium chloride (7 eq) and was purified by chromatography on silica gel (CH₂Cl₂/MeOH) to afford pure compound 31 (20%).

¹H NMR (400 MHz, CDCl₃) δ 8.73 (s, 1H, CHₐᵣ), 7.94 (s, 1H, CH), 4.90 (s, 2H, CH₂), 3.18 (s, 6H, 2 CH₃), 1.46 (s, 9H, 3 CH₃).

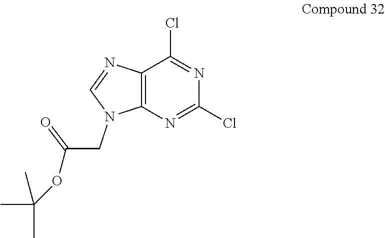 [SMILES: CC(C)(C)OC(=O)Cn1cnc2c(Cl)nc(Cl)nc21]

A suspension of Intermediate Int-4 (25 mg, 0.10 mmol), glycine tert-butyl ester hydrochloride (170 mg, 1.0 mmol) in a mixture of N,N-diisopropylethylamine (0.5 mL) and anhydrous DMF (1 mL) was stirred at room temperature overnight. The mixture was then diluted with ethyl acetate, washed with water, dried over MgSO₄ and concentrated. The residue was purified by chromatography on silica gel (eluent: cyclohexane/ethyl acetate 8/2 to 5/5) to give a mixture compound 32 (23% ratio determined by ¹H NMR) with the corresponding non-cyclized mono-substituted pyrimidine (37%) and N-[2,4-dichloro-6-(dimethylamino)pyrimidin-5-yl]-N′,N′-dimethylimidoformamide (7%).

Compound 32: ¹H NMR (400 MHz, d₆-DMSO) δ 8.13 (s, 1H), 4.93 (s, 2H), 1.42 (s, 9H).

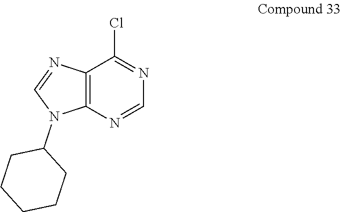 [SMILES: Clc1ncnc2c1ncn2C1CCCCC1]

This compound was synthesised through general synthesis protocol III from 5-amino-4,6-dichloropyrimidine (100 mg, 0.61 mmol), cyclohexylamine and (chloromethylene)dimethyliminium chloride and was purified by chromatography on silica gel (CH₂Cl₂/MeOH) to afford pure compound 33 (64%).

¹H NMR (400 MHz, CDCl₃) δ 8.72 (s, 1H, CHₐᵣ), 8.18 (s, 1H, CHₐᵣ), 4.53 (tt, 1H, J=12.0 Hz, 3.8 Hz), 2.20 (dd, 2H, J=12.7, 2.2 Hz), 1.88 (m, 5H), 1.53 (m, 2H), 1.33 (m, 1H).

1. Method for synthesizing a purine of formula (I) 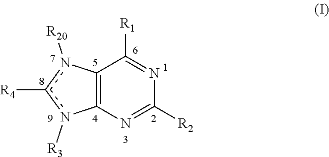
wherein:
the dashed line between the positions 7,8,9 of the imidazole ring symbolizes 2 versions of formula (I): a version v1 wherein there is a double bond between C8 and N7 and hence a radical R₃ on N9 and no radical R₂₀ on N7, as well as a version v2 wherein there is a double bond between C8 and N9 and hence a radical R₂₀ on N7 and no radical R₃ on N9;
R₁ and R₂ are independently selected from the group consisting of hydrogen, halogen, —NR₁₀R₁₁, —N═CR₁₀R₁₁, —OR₁₂, —OSO₂R₁₃, —SOₙR₁₄, —COOR₁₅, —OCOR₁₅, —CONR₁₆R₁₇, alkyl, cycloalkyl, heterocycle, aryl, heteroaryl, alkylaryl, arylalkyl, alkenyl and alkynyl, n being a number between 0 and 2;
R₁₀, R₁₁, R₁₂, R₁₃, R₁₄, R₁₅, R₁₆ and R₁₇ being selected independently from the group consisting of hydrogen, alkyl, cycloalkyl, dialkylamino, heterocycle, aryl, heteroaryl, alkylaryl, arylalkyl, alkenyl and alkynyl;
R₃, if present, is selected from the group consisting of R₁₈ and R₁₉,
R₁₈ being selected from the group consisting of glycosyl,
R₁₉ being selected from the group consisting of hydrogen, alkyl, cycloalkyl, heterocycle, aryl, heteroaryl, alkylaryl, arylalkyl, alkenyl, alkynyl, trialkylsilyl, triarylsilyl, and trialkylarylsilyl;
R₄ is selected from the group consisting of R₆, R₇ and NR₈R₉;
R₆ and R₇ being independently selected from the group consisting of hydrogen, alkyl, cycloalkyl, heterocycle, aryl, heteroaryl, alkylaryl, arylalkyl, alkenyl, alkynyl, halogen, azido, —OR₃₁ and —NR₁₀R₁₁;
R₃₁ being selected from the group consisting of hydrogen, —COR₃₂, —SOₙR₃₃, hydrogen, alkyl, cycloalkyl, heterocycle, aryl, heteroaryl, alkylaryl, arylalkyl, alkenyl, alkynyl, trialkylsilyl, triarylsilyl, and trialkylarylsilyl, n being a number between 0 and 2;
R₈ and R₉ being independently selected from the group consisting of hydrogen, alkyl, cycloalkyl, heterocycle, aryl, heteroaryl, alkylaryl, arylalkyl, alkenyl and alkynyl, or R₈ and R₉ are linked to form a ring;
R₃₂ and R₃₃ being independently selected from the group consisting of hydrogen, alkyl, cycloalkyl, heterocycle, aryl, heteroaryl, alkylaryl, arylalkyl, alkenyl and alkynyl;
R₁₀ and R₁₁ are as defined above;
R₂₀, if present, is selected from the group consisting of R₁₈ and R₁₉,
R₁₈ and R₁₉ are as defined above;
said method consisting in starting from a pyrimidine of formula (II) 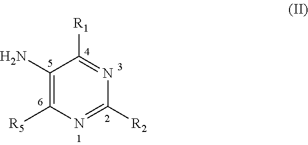
wherein
R₁ and R₂ are as defined above;
R₅ is selected from the group consisting of halogen, NHR₁₀, azido and —OR₃₁, R₁₀ and R₃₁ being as defined above;
said method comprising the following steps a), optionally step b), optionally step c), optionally step d), in any order, and then step e):
a) formation of an amidine group at the C5 or C6 position of the pyrimidine by implementing a Vilsmeier type reagent of formula (III) and/or a reagent of formula (IV) 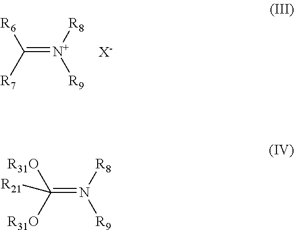
wherein
X⁻ is a counterion;
R₂₁ is selected from the group consisting of R₆ and R₇;
R₆, R₇, R₈, R₉ and R₃₁ are as defined above;
b) optionally, substitution of R₅ by an amine of formula NH₃ or NHR₁₉R₃₄, wherein:
R₁₉ is as defined above,
R₃₄ is selected from the group consisting of hydrogen, —COR₃₅, —SOₙR₃₆, alkyl, cycloalkyl, heterocycle, aryl, heteroaryl, alkylaryl, arylalkyl, alkenyl and alkynyl; and n being a number between 0 and 2;
R₃₅ and R₃₆ being selected from the group consisting of hydrogen, alkyl, cycloalkyl, heterocycle, aryl, heteroaryl, alkylaryl, arylalkyl, alkenyl and alkynyl;
c) optionally, substitution of Y, in a reagent of formula R₁₈Y, by the amino group —NH₂ or —NR₁₉R₃₄ at the C6 position of the pyrimidine, wherein:
R₁₈ is as defined above,
Y is selected from the group consisting of halogen, azido and —OR₃₇, R₃₇ being selected from the group consisting of hydrogen, —COR₃₈, —SOₙR₃₉, alkyl, cycloalkyl, heterocycle, aryl, heteroaryl, alkylaryl, arylalkyl, alkenyl and alkynyl, and n being a number between 0 and 2
R₃₈ and R₃₉ being selected from the group consisting of hydrogen, alkyl, cycloalkyl, heterocycle, aryl, heteroaryl, alkylaryl, arylalkyl, alkenyl and alkynyl;
d) optionally, functionalization of the —NH₂ at the C5 position of the pyrimidine of formula (II), leading to a —NHR₂₀ group, R₂₀ being as defined above;
e) cyclization to form the purine nucleus of formula (I);
with the proviso that when R₅ is NH₂, R₁ is not OH and R₂ is not NH₂;
and wherein, in the above definitions of formulae (I), (II), (III) & (IV), any alkyl, cycloalkyl, heterocycle, aryl, heteroaryl, alkylaryl, arylalkyl, alkenyl or alkynyl group is optionally substituted with one or more substituents selected from the group consisting of alkyl, halogen, haloalkyl, alkoxy, hydroxy, acyl, alkyloxycarbonyl, amino, imino, nitro, —SO₃H, —COOH, —CONH₂, cyano, thiol and oxo.

2. Method according to claim 1, characterized in that R₂ is selected from the group consisting of hydrogen, halogen, preferably —Cl, —NH₂, and alkyl, preferably methyl.

3. Method according to claim 1, characterized in that R₆ or R₇ is a halogen, preferably —Cl.

4. Method according to claim 1, characterized in that R₈ and R₉ are independently selected from the group consisting of hydrogen and alkyl, preferably methyl.

5. Method according to claim 1, characterized in that R₁ is a halogen, preferably —Cl.

6. Method according to claim 1, characterized in that R₃ or R₂₀ is R₁₉, R₁₉ being selected from the group consisting of alkyl, arylalkyl, heteroaryl, and aryl.

7. Method according to claim 1, characterized in that R₃ or R₂₀ is R₁₈, R₁₈ being selected from the group consisting of ribosyl and desoxyribosyl group, preferably, 2′-deoxyribosyl.

8. Method according to claim 1 characterized in that step a) is performed before step b).

9. Method according to claim 1 characterized in that step b) is performed before step a).

10. Method according to claim 1 characterized in that the Vilsmeier type reagent of formula (III) is synthesized in situ.

11. Method according to claim 1 characterized in that step b) is performed using Brønsted or Lewis acid catalyst and/or by heating the reaction mixture.

12. Method according to claim 1 characterized in that the method is performed in one pot with no with no purification of intermediates products.

13. Method according to claim 1 characterized in that steps a) and b) are performed in anhydrous conditions.

14. Intermediate product of the process according to claim 1 selected from the group consisting of:

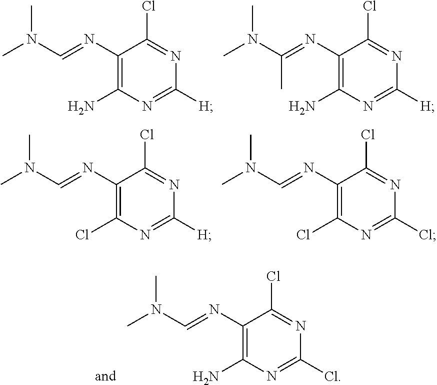

15. Purine selected from the group consisting of 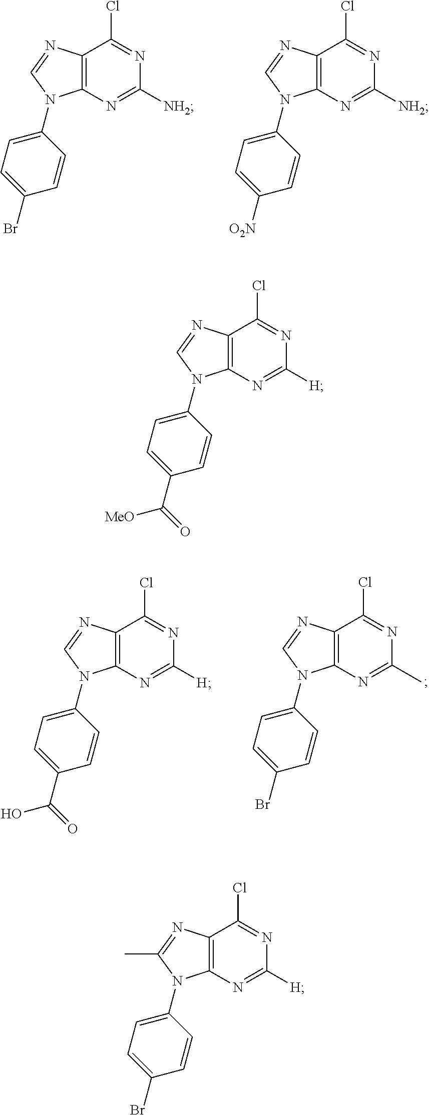 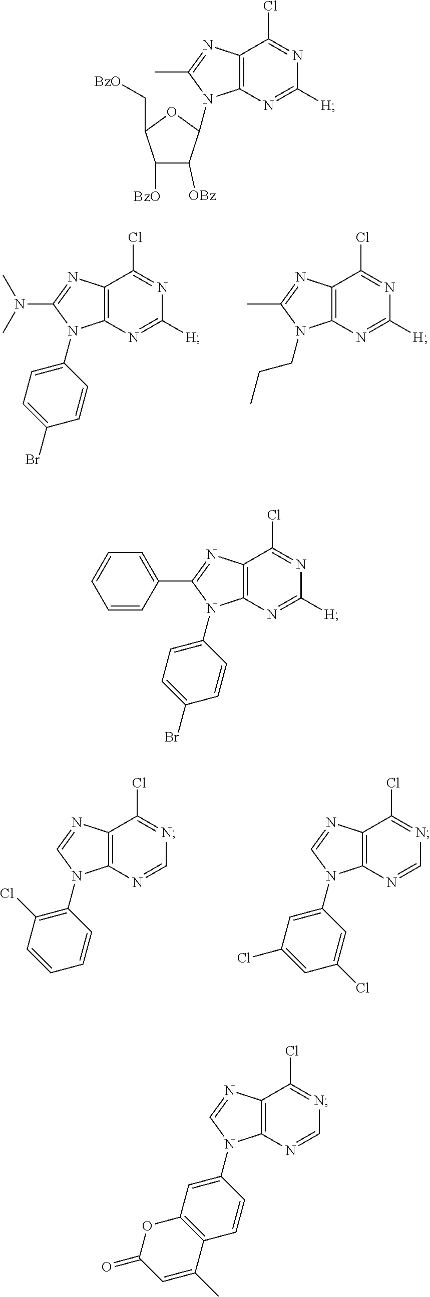 [chemical structure]
